(19) United States
(12) Patent Application Publication
(10) Pub. No.: US 2025/0296928 A1
(43) Pub. Date: Sep. 25, 2025

(54) A SYNTHESIS SCHEME AND PROCEDURES FOR PREPARING A SIK3 INHIBITOR AND INTERMEDIATES THEREOF

(71) Applicant: iOmx Therapeutics AG, Martinsried (DE)

(72) Inventors: GIUSEPPE MARSEGLIA, Verona (IT); LORENZO CARUANA, Verona (IT); TOMMASO CANELLI, Verona (IT); XIANGLIN ZHAO, Shanghai (CN); WENKE WANG, Shanghai (CN); ZIQIANG ZHAO, Shanghai (CN); STEFANIA MANGOLIA, Verona (IT)

(21) Appl. No.: 18/702,686

(22) PCT Filed: Oct. 19, 2022
(86) PCT No.: PCT/EP2022/079131

§ 371 Date: Apr. 18, 2024

(30) Foreign Application Priority Data
Oct. 19, 2021 (EP) 21203530.7

(51) Int. Cl. C07D 417/14; C07D 333/36; C07D 417/12

(52) U.S. Cl. CPC C07D 417/14; C07D 333/36; C07D 417/12

(57) ABSTRACT

The present invention relates to a new scheme and process for the preparation of one specific SIK3 inhibiting compound known as (and described herein as) “E9”. Compound E9 has previously been described by the present applicant to show surprisingly superior drug-like properties, such as in respect of target-potency/specificity and ADMET/PK properties, compared to other prior art SIK3 inhibiting compound. The present invention also relates to novel intermediates used in such process of preparing compound E9, as well as to a new methodology used within the process of producing a characteristic key thiophene-based amino intermediate that is used to produce compound E9, and other related aspects as disclosed herein.

The present invention relates to a new scheme and process for the preparation of one specific SIK3 inhibiting compound known as (and described herein as) “E9”. Compound E9 has previously been described by the present applicant to show surprisingly superior drug-like properties, such as in respect of target-potency/specificity and ADMET/PK properties, compared to other prior art SIK3 inhibiting compound. The present invention also relates to novel intermediates used in such process of preparing compound E9, as well as to a new methodology used within the process of producing a characteristic key thiophene-based amino intermediate that is used to produce compound E9, and other related aspects as disclosed herein.

SIK3 is an intracellular serine/threonine kinase belonging to the AMPK superfamily. Salt-inducible kinases (SIKs) constitute a serine tyrosine kinase subfamily, belonging to the adenosine monophosphate-activated kinase (AMPK) family. Three members (SIK1, -2, and -3) have been identified so far. Amino acid homology of SIK1 with SIK2 and SIK3 is 78% and 68%, respectively, in the kinase domain. The cloning of SIK1 (also known as SIK and SNF1LK), abundantly expressed in the adrenal glands of high-salt, diet-fed rats, led to subsequent cloning of SIK2 (also known as QIK, KIAA0781 and SNF1LK2), mainly expressed in adipose tissues and the rather ubiquitous SIK3 (also known as QSK, KIAA0999 or L19) (Katoh et al. 2004, Mol. Cell. Endocrinol. 217:109). The three SIKs have a similar structure, with an N-terminal kinase domain (catalytic domain), a middle ubiquitin-associated domain (believed important for phosphorylation by LKB1) and a long C-terminal sequence (believed to be a site for further phosphorylation by PKA). However, there are very diverse roles implicated for the various SIKs. For example, various SIKs have been implicated in biological processes as diverse as osteocyte response to parathyroid hormone (Wein et al. 2016, Nature Commun. 7:13176) to induction of SIK1 by gastrin and inhibition of migration of gastric adenocarcinoma cells (Selvik et al. 2014, PLoS ONE 9:e112485). Other potential roles of salt-inducible kinases (in particular SIK3), described in WO2018/193084A1 (to the present applicant, and published 25 Oct. 2018), are furthermore that SIK3 is involved in tumour cell resistance to cell-mediated immune responses, in particular tumour cell resistance to TNF. Recently, SIKs (particularly SIK3) have been demonstrated to also regulate TGFbeta-mediated transcriptional activity and apoptosis, with Hutchinson et al (2010, Cell Death and Disease 11:49) showing that SIK3 expression or activity results in resistance to TGFbeta-mediated apoptosis.

As well as playing a role in various inflammatory responses (Clark et al 2014; Sundberg et al 2016) and oncology—especially the sensitisation of tumour cells to immune responses (WO2018/193084A1)—it has been known since 2011 that inhibition of SIK2 promotes melanogenesis in B16F10 melanoma cells (Kumagai et al 2011, PLoS ONE 6(10): e26148). It was subsequently described that the pigmentation pathway including in human skin explants can be efficaciously induced by (topical) treatment with SIK inhibitors, including those structurally related to YKL-05-099 (Mujahid et al 2017, Cell Reports 19:2177). Indeed, using such results, it has subsequently been sought to claim methods of increasing (the appearance of) skin pigmentation in a subject by administering topically to the subject skin an effective account of a SIK inhibitor (WO2018/160774), including using kinase inhibitors previously known to be SIK inhibitors (WO2016/023014).

Using a high-throughput genetic screening platform (Khandelwal et al, 2015; EMBO molecular medicine 7:450) the applicants have previously reported on the novel role of SIK3 in conferring TNF resistance to tumour cells (WO2018/193084A1; Michels et al, 2019; Abstract A184 CICON, Paris 25-28 Sep. 2019). SIK3 mediates this effect by retaining HDAC4 in the cytoplasm via direct phosphorylation, which MHC potentiates the nuclear activity of pro-tumorigenic transcription factor NFKB in response to TNF.

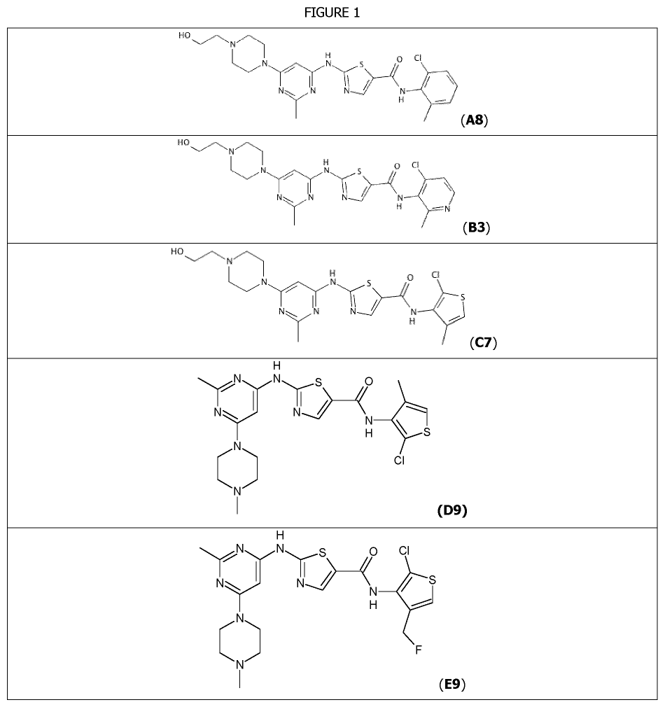

The applicants recently reported (WO2020/083926; Michels et al, 2020; Cancer Res 80 (AACR Suppl 16):Abstract 6698) on the development of a first-in-class, potent, oral inhibitor of SIK3 described as “C7” (FIG. 1). Compound C7 effectively engages the SIK3 pathway in tumour cells by inhibiting the phosphorylation of its substrate HDAC4 and abrogating the TNF-induced transcriptional activity of NFKB in a dose-dependent manner both in vitro and in vivo. As a result, C7 makes tumour cells, both human and murine, increasingly sensitive to TNF-mediated lysis while sparing healthy PBMCs in vitro. Treatment of established tumours in different syngeneic tumour mouse models (MC38, EMT6) with C7 resulted in significant tumour growth inhibition in monotherapy setting. The effect was comparable or even superior to anti-PD-1 treatment alone. Furthermore, immune cell profiling of treated mice showed a significant infiltration of activated T cells, along with remarkable reduction in immunosuppressive Tregs and M2 TAMs. Given the emerging role of TNF resistance in tumour immune evasion and known dependency on NFKB pathway by multiple solid tumours, SIK3 inhibitors that work by abrogating TNF-driven NFKB activity merit further investigation in the clinics for the treatment of cancer.

As is described by the present applicant in co-pending PCT/EP2021/060338 (unpublished on the priority date of the present invention), and as reiterated within the comparative Examples 1 to 5 of the present disclosure, a superior SIK3 inhibiting compound named “E9” (FIG. 1) was found to possess surprisingly improved drug-like properties over the structurally-similar thiophene compound C7, as well as over other structurally-related compounds D9, B3 and A8 (FIG. 1).

The synthesis of compound E9, and that of a characteristic thiophene-based key intermediate known as compound “46”, are specifically described in PCT/EP2021/060338. The synthesis schemes and procedures for compounds E9 and 46 described therein (and reiterated in comparative Example 6 of the present disclosure) are suitable, for example, for “research-grade” and/or “bench-scale” preparation of such compounds.

However, the high-suitability of compound E9 as a drug-candidate and, potentially as an approved drug, requires schemes and procedures for this synthesis that are more suitable for larger-scale production of E9, such as at a batch-scale of greater than about 125 g, in order to prepare efficiently (in terms of cost and/or time) compound E9 to enable clinical trials and, potentially, commercial availability. There is, therefore, a need to provide new and/or improved procedures (such as schemes or processes) to prepare compound E9, as well as intermediate compounds that are used to prepare compound E9.

Accordingly, it is one object of the present invention to provide a procedure to prepare compound E9 (and/or intermediates used in the preparation of E9) that overcomes one or more of these problems, such as a new procedure to prepare compound E9 that reduces the overall cost or time and/or that improves the overall yield of E9 (eg, in absolute terms or in terms of the amount of key intermediate(s) needed to prepare a unit amount of E9), and/or a procedure that improves purity and/or yield of such key intermediates. An object underlying the present invention is solved by the subject matter as disclosed or defined anywhere herein, for example by the subject matter of the attached claims.

Generally, and by way of brief description, the main aspects of the present invention can be summarised as follows:

In a first aspect, the present invention provides a method of preparing the compound E9

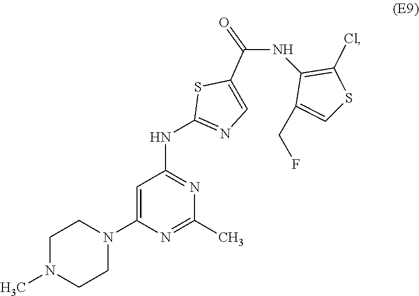 [SMILES: Cc1nc(Nc2ncc(C(=O)Nc3c(CF)csc3Cl)s2)cc(N2CCN(C)CC2)n1]

wherein the method comprises a step of reacting under coupling conditions a carboxylic acid intermediate having the formula I:

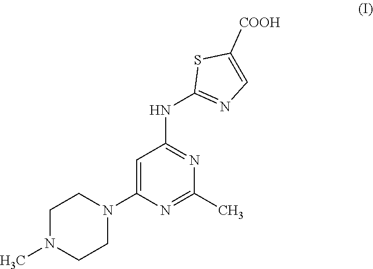 [SMILES: Cc1nc(Nc2ncc(C(=O)O)s2)cc(N2CCN(C)CC2)n1]

with an amino intermediate having the formula II:

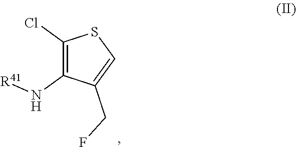

wherein R⁴¹ is selected from the group consisting of H or an amino protecting group.

In a second aspect, the present application provides a method of preparing an amino intermediate useful for preparing the compound E9

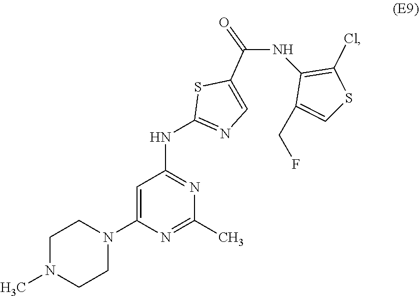 [SMILES: Cc1nc(Nc2ncc(C(=O)Nc3c(CF)csc3Cl)s2)cc(N2CCN(C)CC2)n1]

wherein the amino intermediate is 46

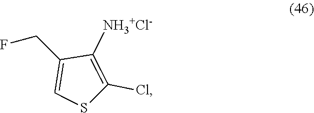

wherein the method comprises a step of reacting compound 45

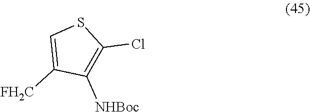

with HCl and ethyl acetate (EA).

In a third aspect, the present application provides an intermediate useful for preparing the compound E9

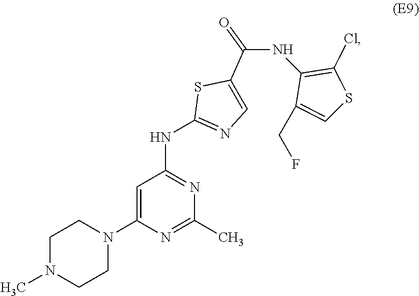 [SMILES: Cc1nc(Nc2ncc(C(=O)Nc3c(CF)csc3Cl)s2)cc(N2CCN(C)CC2)n1]

wherein the intermediate is a carboxylic acid intermediate having the formula I:

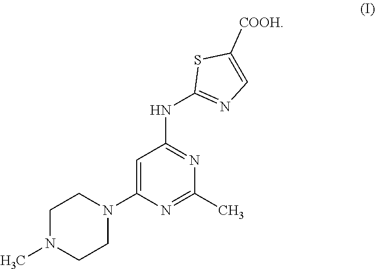 [SMILES: Cc1nc(Nc2ncc(C(=O)O)s2)cc(N2CCN(C)CC2)n1]

In a fourth aspect, the present application provides an intermediate useful for preparing the compound E9

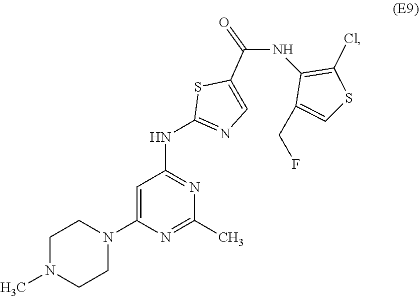 [SMILES: Cc1nc(Nc2ncc(C(=O)Nc3c(CF)csc3Cl)s2)cc(N2CCN(C)CC2)n1]

wherein the intermediate is an ester intermediate having the formula Ia:

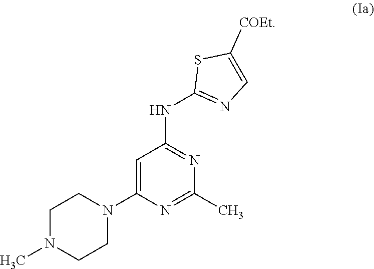

In a fifth aspect, the present application provides a bulk amount of a compound, wherein the compound is one and, in the respective amount, selected from the group consisting of:
(A) at least about 125 g of compound E9

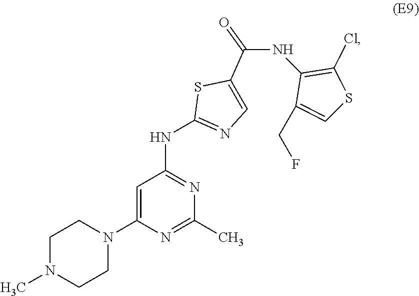 [SMILES: Cc1nc(Nc2ncc(C(=O)Nc3c(CF)csc3Cl)s2)cc(N2CCN(C)CC2)n1]

(B) at least about 125 g of the carboxylic acid intermediate having the formula I:

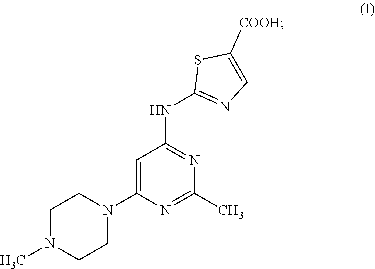 [SMILES: Cc1nc(Nc2ncc(C(=O)O)s2)cc(N2CCN(C)CC2)n1]

(C) at least about 125 g of the ester intermediate having the formula Ia:

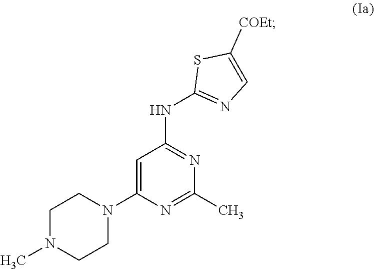

(D) at least about 2.5 g of the amino intermediate 46

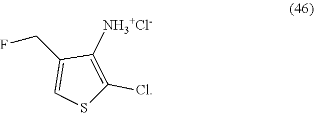

The figures show:

FIG. 1: depicts the structure of compound E9, a potent inhibitor of SIK3 and SIK2, as described in PCT/EP2021/060338 (unpublished on the priority date of the present application). Also depicted is the structure of the multi kinase-inhibiting approved drug dasatinib (A8), and the structures of other published prior-art SIK3 inhibiting compounds B3 (WO2018/193084), C7 and D9 (both, WO2020/083926).

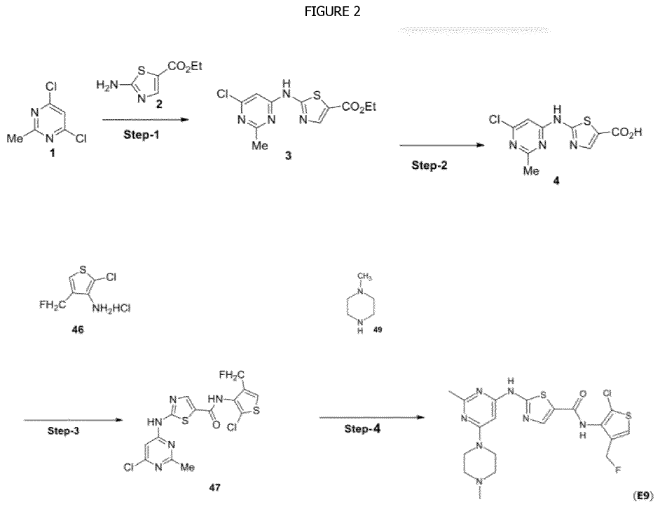

FIG. 2: depicts the synthesis scheme for compound E9 as described in PCT/EP2021/060338 (unpublished on the priority date of the present application).

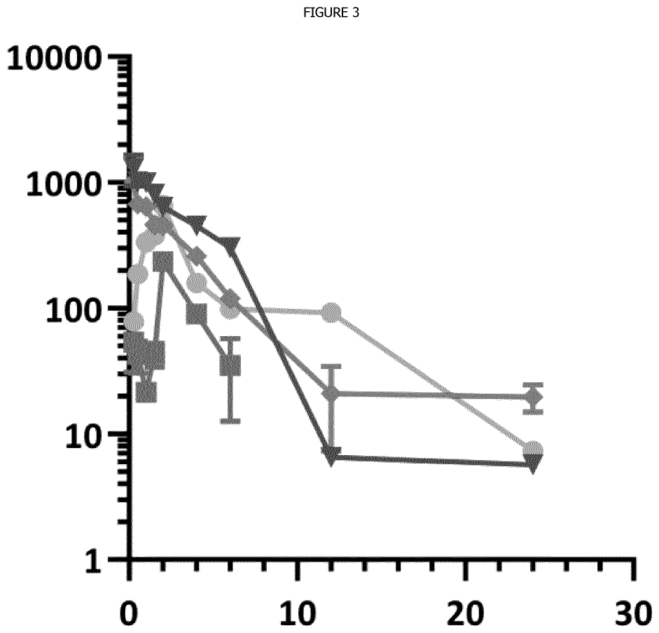

FIG. 3: depicts pharmacokinetic curves of compounds of formula (Ia) E9 (inverted triangles), and E10 (diamonds), compared to a closely related compound C7 (squares) following 30 mg/kg po administration. Y axis=Total plasma compound concentration (ng/ml); X axis=Time (h). Other compounds disclosed in PCT/EP2021/060338 are described by circles (E4) and diamonds (E10).

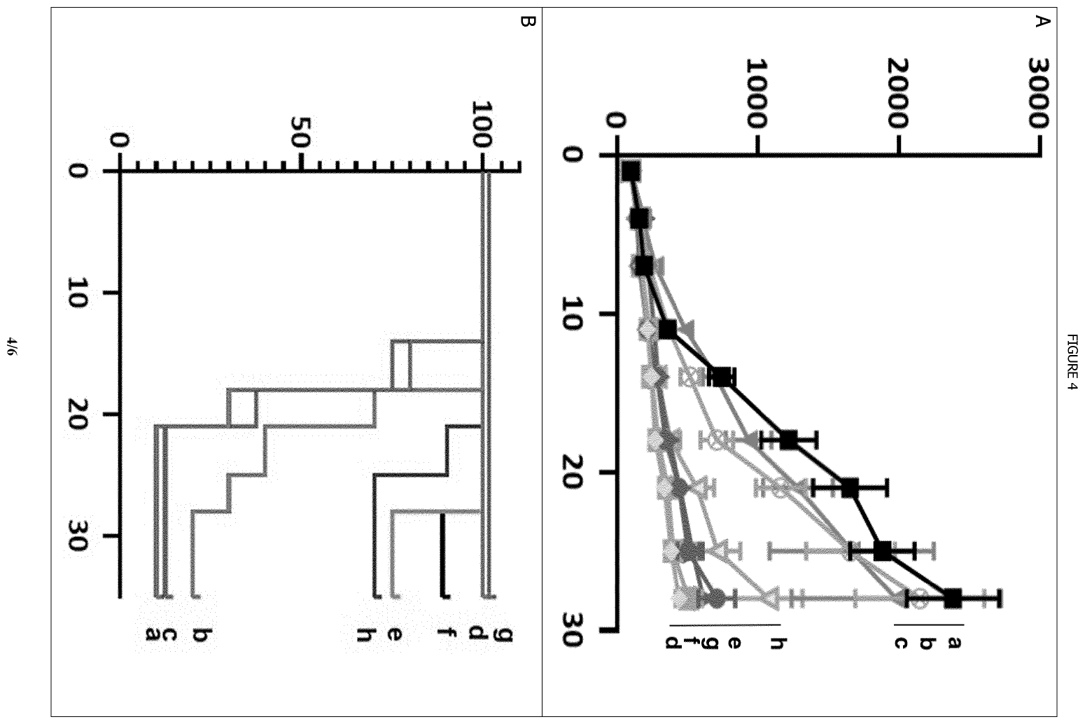

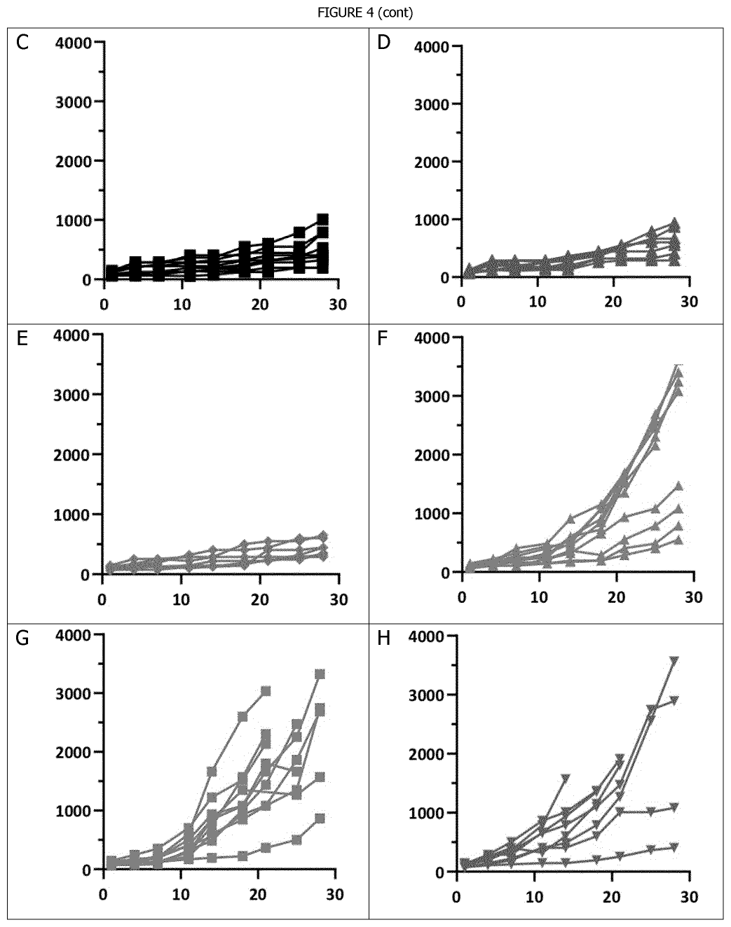

FIG. 4: depicts (A) tumour growth kinetics in mice implanted with MC38 cells, and upon treatment with: (1) controls: ratIgG2a 10 mg/kg (black filled squares), aPD-1 10 mg/kg 3q7d (light grey open crossed-circles), and vehicle (light grey filled inverted triangles); and compounds E10 30 mg/kg BID (grey open inverted triangles), E4 40 mg/kg BID (dark grey filled circles), E9 25 mg/kg BID (light grey filled squares), E9 50 mg/kg BID (dark grey filled triangles) and C7 100 mg/kg BID (light grey filled diamonds). Y-axis=Mean tumour volume (mm3). Error bars SEM Statistical significance was calculated with one-way ANOVA analysis including Tukey's multiple comparison analysis. X-axis=Days. (B) Probability of tumour volume <1000 mm3 of the mice in (A). Y-axis: Probability of occurrence of tumour volumes <=1000 mm3 upon treatment. X-axis: Days. For both (A) and (B), Controls: ratIgG2a (a), aPD-1 10 mg/kg (b), vehicle (c); and compounds: C7 100 mg/kg (d) BID, E4 40 mg/kg BID (e), E9 25 mg/kg BID (f), E9 50 mg/kg BID (g), and E10 30 mg/kg BID (h). (C) to (H) Tumour growth curves of individual mice; Y-axis: Tumour volume (mm3), X-axis: Days. (C) E9 25 mg/kg BID; (D) E9 50 mg/kg BID; (E) C7 100 mg/kg BID; (F) aPD-1 10 mg/kg; (G) ratIgG2a 10 mg/kg and (H).

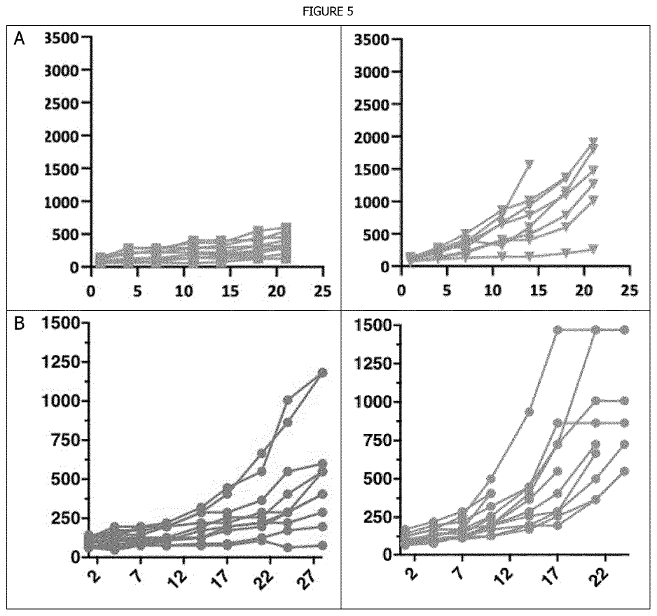

FIG. 5: depicts superior and more uniform tumour growth inhibition in a MC38 syngeneic tumour model by compound E9 (24 mg/kg BID) (A), compared to dasatinib (A8, 30 mg/kg QD) (B). Left hand column=compound treatment group; and right hand column=vehicle treatment group. Y axes=Tumour volume (mm3); X axes=Days after inoculation.

The present invention, and particular non-limiting aspects and/or embodiments thereof, can be described in more detail as follows.

Although the present invention may be further described in more detail, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by what is described, defined or otherwise disclosed herein, in particular in any itemised embodiments or the appended claims.

Herein, certain elements of the present invention are described in more detail. These elements are listed with specific embodiments; however, it should be understood that they may be combined in any manner and in any number to create additional embodiments. The variously described examples and preferred embodiments should not be construed to limit the present invention to only the explicitly described embodiments. This description of this application should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise. For example, if in one embodiment of a method of the invention a reaction is conducted for about 3 h and in another embodiment of such method of the invention the reaction is conducted between 5 and 25° C., then in a preferred embodiment of such method of the invention, the reaction is conducted for about 3 h at between 5 and 25° C.

Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present invention will employ, unless otherwise indicated, conventional methods of chemistry, biochemistry, and recombinant DNA techniques which are explained in the literature in the field (cf., e.g., Molecular Cloning: A Laboratory Manual, 2^{nd} Edition, 1 Sambrook et al. eds., Cold Spring Harbor Laboratory Press, Cold Spring Harbor 1989).

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The term “consisting essentially of” means excluding other members, integers or steps of any essential significance or group of members, integers or steps of any essential significance. For example, a pharmaceutical composition consisting essentially of the members/components as defined herein (such as a compound as defined in any of the aspects of the invention and optionally one additional therapeutic agent) would exclude further therapeutic agents (besides the compound as defined in any of the aspects of the invention and the optional one additional therapeutic agent) but would not exclude contaminants (e.g., those from the isolation and purification method) in trace amounts (e.g., the amount of the contaminant (preferably the amount of all contaminants present in the composition) is less than 5% by weight, such as less than 4% by weight, 3% by weight, 2% by weight, 1% by weight, 0.5% by weight, 0.4% by weight, 0.3% by weight, 0.2% by weight, 0.1% by weight, 0.05% by weight, with respect to the total composition) and/or pharmaceutically acceptable excipients (such as carriers, e.g., phosphate buffered saline, preservatives, and the like). The term “consisting of” means excluding all other members, integers or steps of significance or group of members, integers or steps of significance. For example, a pharmaceutical composition consisting of the members/components as defined herein (such as a compound as defined in any of the aspects of the invention, one excipient, and optionally one additional therapeutic agent) would exclude any other compound (including a second or further excipient) in an amount of more than 2% by weight (such as any other compound in an amount of more than 1% by weight, more than 0.5% by weight, more than 0.4% by weight, more than 0.3% by weight, more than 0.2% by weight, more than 0.1% by weight, more than 0.09% by weight, more than 0.08% by weight, more than 0.07% by weight, more than 0.06% by weight, more than 0.05% by weight, more than 0.04% by weight, more than 0.03% by weight, more than 0.02% by weight, more than 0.01% by weight) with respect to the total composition. The term “comprising” encompasses the term “consisting essentially of” which, in turn, encompasses the term “consisting of”. Thus, at each occurrence in the present application, the term “comprising” may be replaced with the term “consisting essentially of” or “consisting of”. Likewise, at each occurrence in the present application, the term “consisting essentially of” may be replaced with the term “consisting of”.

Where used herein, “and/or” is to be taken as specific disclosure of each of the two specified features or components with or without the other. For example, “X and/or Y” is to be taken as specific disclosure of each of (i) X, (ii) Y, and (iii) X and Y, just as if each is set out individually herein.

In the context of the present invention, the terms “about” and “approximately” are used interchangeably and denote an interval of accuracy that the person of ordinary skill will understand to still ensure the technical effect of the feature in question. The term typically indicates deviation from the indicated numerical value by ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, and for example ±0.01%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect.

The terms “a”, “an” and “the” and similar references used in the context of describing the invention (especially in the context of the claims) are to be construed to cover both the singular and the plural, unless otherwise indicated herein or clearly contradicted by the context.

Recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by the context.

The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Several documents are cited throughout the text of this specification. Each of the documents cited herein (including all patents, patent applications, scientific publications, manufacturer's specifications, instructions, etc.), whether supraor infra, are hereby incorporated by reference in their entirety. Nothing herein is to be construed as an admission that the invention is not entitled to antedate such disclosure by virtue of prior invention.

The terms “of the [present] invention”, “in accordance with the [present] invention”, “according to the [present] invention” and the like, as used herein are intended to refer to all aspects and embodiments of the invention described and/or claimed herein.

It is to be understood that the application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above or below are not limited to any particular aspect or embodiment of the invention and apply equally to all aspects and embodiments that are described.

The term “impurity” as used herein refers to any foreign material (in particular chemical substances) which may be present in a composition comprising a desired compound (e.g., a composition comprising a compound described herein, such a compound E9. Impurities may occur naturally, may be added during the synthesis and/or purification of the desired compound, or may be generated during the synthesis and/or purification of the desired compound. Exemplary impurities include one or more starting materials, one or more solvents, one or more intermediates or reactants, one or more degradation products of any of the foregoing or of the desired compound, one or more leftovers of protecting groups after deprotection, and combinations thereof.

The term “optional” or “optionally” as used herein means that the subsequently described event, circumstance or condition may or may not occur, and that the description includes instances where said event, circumstance, or condition occurs and instances in which it does not occur.

The expression “amino protecting group” as used herein preferably refers to any group by which an amino group contained in a compound can be transferred into a less reactive (i.e., protected) amino group. Preferably, amino protecting groups can be incorporated into the corresponding compound under mild conditions, in a chemoselective and/or regioselective manner, and/or in good yields. Furthermore, the amino protecting groups should be stable under the conditions to which the protected compound is to be subjected (e.g., the conditions of the desired reaction and/or purification conditions). Preferably, the amino protecting groups should minimize the risk of racemization of a stereogenic center, when present in the compound. In one embodiment, the amino protecting groups should be removable from the protected compound under mild conditions and in a selective manner such that the deprotected compound is obtained in high yields. Exemplary amino protecting groups include tert-butyloxycarbonyl (BOC), 9-fluorenylmethoxycarbonyl (FMOC), benzyloxycarbonyl (Cbz), p-methoxybenzylcarbonyl (MOZ), acetyl (Ac), trifluoroacetyl, benzoyl (Bz), benzyl (Bn), p-methoxybenzyl (PMB), 3,4-dimethoxyphenyl (DMPM), p-methoxyphenyl (PMP), 2,2,2-trichloroethoxycarbonyl (Troc), triphenylmethyl (trityl; Tr), toluenesulfonyl (tosyl; Ts), para-bromophenylsulfonyl (brosyl), 4-nitrobenzenesulfonyl (nosyl), and 2-nitrophenylsulfenyl (Nps). Other Exemplary amino protecting groups include allyloxycarbonyl (alloc) and phthalimide.

The term “half-life” relates to the period of time which is needed to eliminate half of the activity, amount, or number of molecules. In the context of the present invention, the half-life of a compound disclosed herein (eg compound E9, or C7) is indicative for the stability of said compound.

The terms “subject”, “patient”, “individual”, or “animal” relate to multicellular animals, such as vertebrates. For example, vertebrates in the context of the present invention are mammals, birds (e.g., poultry), reptiles, amphibians, bony fishes, and cartilaginous fishes, in particular domesticated animals of any of the foregoing as well as animals (in particular vertebrates) in captivity such as animals (in particular vertebrates) of zoos. Mammals in the context of the present invention include, but are not limited to, humans, non-human primates, domesticated mammals, such as dogs, cats, sheep, cattle, goats, pigs, horses etc., laboratory mammals such as mice, rats, rabbits, guinea pigs, etc. as well as mammals in captivity such as mammals of zoos. The term “animal” as used herein also includes humans. Particular non-limiting examples of birds include domesticated poultry, and include birds such as chickens, turkeys, ducks, geese, guinea fowl, pigeons, pheasants etc.; while particular non-limiting examples of bony or cartilaginous fish include those suitable for cultivation by aquiculture, and include bony fish such as salmon, trout, perch, carp, cat-fish, etc.

The compound dasatinib (N-(2-chloro-6-methylphenyl)-2-[[6-[4-(2-hydroxyethyl)-1-piperazinyl]-2-methyl-4-pyrimidinyl]amino]-5-thiazolecarboxamide, in particular, in monohydrate form; and herein also referred to as compound A8) has the following structure:

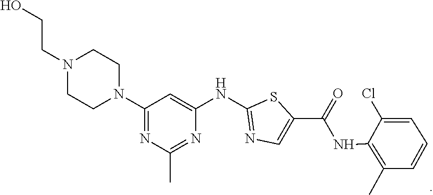 [SMILES: Cc1nc(Nc2ncc(C(=O)Nc3c(C)cccc3Cl)s2)cc(N2CCN(CCO)CC2)n1]

In a first aspect, the present invention provides a method of preparing the compound E9

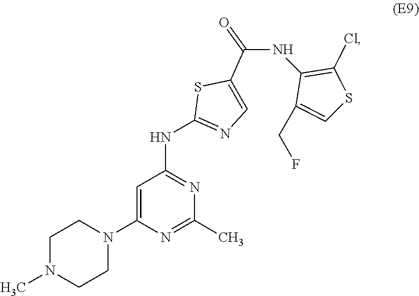 [SMILES: Cc1nc(Nc2ncc(C(=O)Nc3c(CF)csc3Cl)s2)cc(N2CCN(C)CC2)n1]

wherein the method comprises a step of reacting under coupling conditions a carboxylic acid intermediate having the formula I:

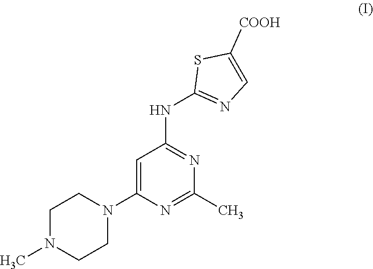 [SMILES: Cc1nc(Nc2ncc(C(=O)O)s2)cc(N2CCN(C)CC2)n1]

with an amino intermediate having the formula II

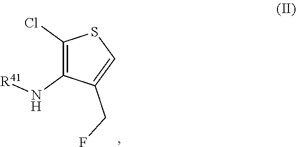

wherein R⁴¹ is selected from the group consisting of H or an amino protecting group.

Compound E9 (see below) is described by the present applicant in co-pending PCT/EP2021/060338 (unpublished on the priority date of the present invention), as a SIK3 inhibiting compound found to possess surprisingly improved drug-like properties over the structurally-similar thiophene compound C7, as well as over other structurally-related compounds D9, B3 and A8 (FIG. 1).

| | | |
|---|---|---|
| E9 | 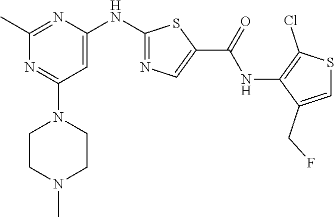 [SMILES: Cc1nc(Nc2ncc(C(=O)Nc3c(CF)csc3Cl)s2)cc(N2CCN(C)CC2)n1] | N-(2-chloro-4-(fluoromethyl)thiophen- 3-yl)-2-((2-methyl-6-(4- methylpiperazin-1-yl)pyrimidin-4- yl)amino)thiazole-5-carboxamide |

In one embodiment of the method of the first aspect, is a method of preparing an amount of compound E9, or in other aspects of the invention is a method of preparing an amount of carboxylic acid intermediate, or an amount of ester immediate Ia, that (in each case) is a bulk amount, such as in an amount that is greater than about: 1.5 g, about 2.5 g, about 5 g, about 15 g, about 25 g, about 40 g, or about 75 g, in particular for preparing an amount of compound that is greater than about 125 g. For example, such method may prepare an amount of a compound (eg E9, I or Ia) that is greater than an amount selected from the group consisting of: about 150 g, about 200 g, about 250 g, about 400 g, about 600 g, about 750 g, and about 800 g. Indeed, a method of the invention may be used to prepare greater amounts of compound, such as greater than about 1.0 kg of such compound, in particular, to prepare more than about 1.25 kg, about 1.5 kg, about 1.75 kg or about 2 kg of the compound. In certain embodiments, the amount of compound prepared may be an amount of up to about 1.0 kg, 2.0 kg, 5 kg or more, such as up to about 10 kg, or more, of a compound. In one particular embodiment, of the methods of the invention is a method of preparing an amount of compound that is between 250 g and 25 kg of such compound, such as between 400 g and 4.0 kg of compound. Any of such amounts (or ranges of amounts) of compound as described in this paragraph, for example, when applied to an amount of compound E9, or to carboxylic acid intermediate I or to ester immediate Ia, can be described as a “bulk amount” of such compound.

Such an amount of compound E9 may be prepared (or contained) in one or more vessels (eg reaction vessels), containers or receptacles. In one particular such embodiment, the method of the first aspect of the invention, is used to prepare such an amount of compound E9 in a single (eg, reaction) vessel (such as a jacketed reactor).

In one embodiment of the method of the first aspect of the invention, the method is used to prepare a (eg, substantially) pure form of compound E9.

A compound referenced herein (e.g., compounds E9, or 46, or those having the formula I or formula Ia) can, in certain embodiments, be in (e.g., prepared as, or provided in) a purified or (e.g., substantially) pure form. For example, the compound may be greater than about 50% pure, such as greater than about 60%, 70% or 80% pure, suitably greater than about 90% pure (in particular, greater than about 95%, 97% 98% and even 99%). That is, in certain of such embodiments such a compound is present together with only a limited amount of impurities (e.g., such as those introduced during manufacturing), such as only small amounts of impurities are present, including embodiments where the compound is present in a form where impurities are substantially absent. The purity (e.g., the absence, or degree of presence of impurities) of the compound can be determined by routine procedures e.g. by HLPC.

In certain embodiments of the respective aspect of the present invention, a “pure form” of the applicable compound may be one containing less than about 50%, 40%, 30% and suitably 10% or 5% area by HPLC of total impurities, preferably less than about 3% and 2% area by HPLC of total impurities. In particular, a pure form of the applicable compound may be one that contains less than 2% or 1% of total impurities (eg, as may be determined by area by HPLC). The term “% area by HPLC” as used herein refers to the area in an HPLC chromatogram of one or more peaks compared to the total area of all peaks in the HPLC chromatogram expressed in percent of the total area. Further, the purity of the compound may be expressed herein as “HPLC” purity. As such, “HPLC purity”, is a calculation of the area under the compound peak divided by the total area under the curve in an HPLC chromatogram. Suitably, the compound contains less than about 10% area by HPLC of total impurities. More preferably, less than about 5% area by HPLC of total impurities.

In one embodiment of the method of the first aspect, R⁴¹ is an amino protecting group and the amino protecting group is selected from the group consisting of: tert-butyloxycarbonyl (BOC), 9-fluorenylmethoxycarbonyl (FMOC), benzyloxycarbonyl (Cbz), p-methoxybenzylcarbonyl (MOZ), acetyl (Ac), trifluoroacetyl, benzoyl (Bz), benzyl (Bn), p-methoxybenzyl (PMB), 3,4-dimethoxyphenyl (DMPM), p-methoxyphenyl (PMP), 2,2,2-trichloroethoxycarbonyl (Troc), triphenylmethyl (trityl; Tr), toluenesulfonyl (tosyl; Ts), para-bromophenylsulfonyl (brosyl), 4-nitrobenzenesulfonyl (nosyl), and 2-nitrophenylsulfenyl (Nps). In another embodiment of the method of the first aspect, R⁴¹ is an amino protecting group and the amino protecting group is selected from the group consisting of: allyloxycarbonyl (alloc), and phthalimide.

In one embodiment of the method of the first aspect, the amino intermediate having formula II is compound 46

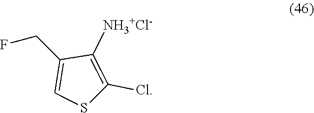

In one embodiment of the method of the first aspect, the reaction of the amino intermediate II with the carboxylic acid intermediate I is conducted in the presence of a coupling agent and/or a base.

In one embodiment of the method of the first aspect, the reaction of the amino intermediate II with the carboxylic acid intermediate I takes place in a solvent, such as an aprotic solvent, e.g., acetonitrile. Thus, in a preferred embodiment of the first aspect, the method comprises reacting of the amino intermediate II with the carboxylic acid intermediate I in a solvent in the presence of (i) a base and/or (ii) a coupling agent.

It is within the skills of a skilled person to determine suitable reaction parameters (eg amounts of the amino intermediate II and the carboxylic acid intermediate I, optionally, the amounts of base and/or coupling agent, and/or solvent; reaction temperature; reaction time; etc.) in order to obtain the desired product. Preferably, an excess of base relative to the molar amount of carboxylic acid is used (eg the base is used in at least about 1.5 eq., such as at least about 2 eq., at least about 2.5 eq., at least about 3 eq., at least about 3.5 eq., or at least about 4 eq., and up to about 5 eq., the molar amount of the carboxylic acid). Furthermore, the intermediate and the carboxylic acid may be reacted in a molar ratio of intermediate to carboxylic acid of about 0.8:1 to about 1:1.2, such as about 0.9:1 to about 1:1.1, or in about equimolar amounts. The amount of coupling agent when used is preferably about at least about 1.0 eq., such as at least about 1.1 eq., at least about 1.2 eq., at least about 1.3 eq., and up to about 2 eq., the molar amount of the carboxylic acid.

Also within the skills of a skilled person is to determine a suitable bases and/or coupling agent for the amide body formation of the reaction. For example, the skilled person may refer to text books or to reviews such as the review of Valeur and Bradley 2009 (Chem Soc Rev 38:606).

In one embodiment of the method of the first aspect, the coupling agent is selected from the group consisting of: N,N,N′,N′-tetramethylchloroformamidinium hexafluorophosphate (TCFH), 1,3-dimethyl-2-chloro-4,5-dihydro-1H-imidazolium hexafluorophosphate (DCIH) N,N,N′,N′-tetramethylfluoroformamidinium hexafluorophosphate (TFFH), 1-ethyl-3-(3-dimethylaminopropyl)carbodiimide (EDCI), dicyclohexylcarbodiimide (DCC), diisopropylcarbodiimide (DIC), 1-[bis(dimethylamino)methylene]-1H-1,2,3-triazolo[4,5-b]pyridinium 3-oxide hexafluorophosphate (HATU), 2-(1H-benzotriazol-1-yl)-1,1,3,3-tetramethyluronium hexafluorophosphate (HBTU), 2-(1H-benzotriazol-1-yl)-1,1,3,3-tetramethylaminium tetrafluoroborate (TBTU), 1-propanephosphonic anhydride (T3P), benzotriazol-1-yl-oxytripyrrolidinophosphonium hexafluorophosphate (PyBOP), 7-azabenzotriazol-1-yloxy)tripyrrolidinophosphonium hexafluorophosphate (PyAOP), and (6-chlorobenzotriazol-1-yloxy)tripyrrolidinophosphonium hexafluorophosphate (TPTDP), especially selected from the group consisting of: N,N,N′,N′-tetramethylchloroformamidinium hexafluorophosphate (TCFH), 1,3-dimethyl-2-chloro-4,5-dihydro-1H-imidazolium hexafluorophosphate (DCIH) and N,N,N′,N′-tetramethylfluoroformamidinium hexafluorophosphate (TFFH). In one particular such embodiment, the coupling agent is N,N,N′,N′-tetramethylchloroformamidinium hexafluorophosphate (TCFH).

In one embodiment of the method of the first aspect, the base is a non-nucleophilic base, such as a non-nucleophilic base selected from the group consisting of 1-methylimidazole (NMI), 4-dimethylaminopyridine (DMAP), N,N-diisopropylethylamine (DiPEA), 2,2,6,6-tetramethylpiperidine, trimethylamine (TEA), tributylamine, 1,8-diazabicycloundec-7-ene (DBU), 1,5-diazabicyclo[4.3.0]non-5-ene (DBN), 1,5,7-triazabicyclo[4.4.0]dec-5-ene (TBD), 7-methyl-1,5,7-triazabicyclo[4.4.0]dec-5-ene (MTBD), 1,4-diazabicyclo[2.2.2]octane (TED), collidine, 1,1,3,3-tetramethylguanidine (TMG), quinuclidine, 2,2,6,6-tetramethylpiperidine (TMP), pempidine (PMP), 2,6-di-tert-butylpyridine, 2,6-lutidine, phosphazene bases (eg t-Bu-P₄), lithium diisopropylamide (LDA), sodium bis(trimethylsilyl)amide (NaHMDS), potassium bis(trimethylsilyl)amide (KHMDS), sodium tert-butoxide, and potassium tert-butoxide; especially selected from the group consisting of: 1-methylimidazole (NMI), 4-dimethylaminopyridine (DMAP), N,N-diisopropylethylamine (DiPEA) and trimethylamine (TEA). In one particular such embodiment, the base is N,N-diisopropylethylamine (DiPEA).

In one embodiment of the method of the first aspect, the reaction comprises: reacting about 1 molar-equivalent (“Equiv.” or “eq.”) of the carboxylic acid intermediate having formula I:

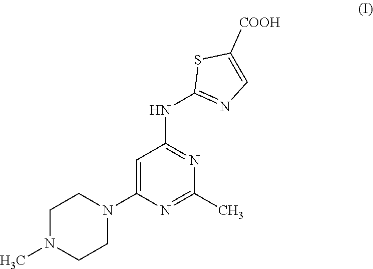 [SMILES: Cc1nc(Nc2ncc(C(=O)O)s2)cc(N2CCN(C)CC2)n1]

with between 0.5 and 5 Equiv. of the amino intermediate 46

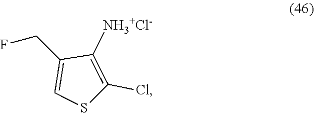

in the presence of between 0.8 and 7 Equiv. of tetramethylchloroformamidinium hexafluorophosphate (TCFH) and between 0.2 and 20 Equiv. of N,N-diisopropylethylamine (DiPEA).

In one embodiment of the method of the first aspect, each about 1 Equiv. of the carboxylic acid intermediate having formula I is reacted with an Equiv. amount of the amino intermediate 46 selected from the group consisting of: between 1 and 4 Equiv., between 1 and 3 Equiv., between 1 and 2 Equiv. and between 1 and 1.8 Equiv., especially selected from the group consisting of: between 1 and 1.5 Equiv., between 1 and 1.4 Equiv. and between 1 and 1.3. In one particular such embodiment, each about 1 Equiv. of the carboxylic acid intermediate having formula I is reacted with between 1.1 and 1.2 Equiv. of the amino intermediate 46.

In one embodiment of the method of the first aspect, each about 1 Equiv. of the carboxylic acid intermediate having formula I is reacted in the presence of an Equiv. amount of tetramethylchloroformamidinium hexafluorophosphate (TCFH) selected from the group consisting of: 1 to 6 Equiv., 1 to 5 Equiv., 1 to 4 Equiv., 1 to 3 Equiv., 1 to 2.5 Equiv., 1 to 2 Equiv., and 1 to 1.9 Equiv., especially selected from the group consisting of: 1.1 to 1.8 Equiv., 1.2 to 1.7 Equiv., and 1.3 to 1.6 Equiv. In one particular such embodiment, each about 1 Equiv. of the carboxylic acid intermediate having formula I is reacted in the presence of between 1.4 and 1.5 Equiv. of tetramethylchloroformamidinium hexafluorophosphate (TCFH).

In one embodiment of the method of the first aspect, the carboxylic acid intermediate having formula I, the amino intermediate 46 and the tetramethylchloroformamidinium hexafluorophosphate (TCFH) are mixed in a polar aprotic solvent to form a mixture and the N,N-diisopropylethylamine (DiPEA) is added subsequently to the mixture; suitably wherein the N,N-diisopropylethylamine (DiPEA) is added subsequently to the mixture in limited amounts over a period of time, such as dropwise.

A polar solvent may be a solvent having a dipole moment of at least 1.4 D, especially a dipole moment from 1.4 D to 5.0 D. An aprotic solvent s a solvent that lacks an acidic proton, eg lacks hydroxyl and amine groups, and, therefore, does not act as a proton donor in hydrogen bonding.

In one embodiment of the method of the first aspect, the polar aprotic solvent is selected from the group consisting of acetone, acetonitrile, dichloromethane, dimethylformamide, dimethylpropyleneurea, dimethylsulfoxide, ethyl acetate, hexamethylphosphoric triamide, and tetrahydrofuran. In one particular such embodiment, the polar aprotic solvent is acetonitrile.

In one embodiment of the method of the first aspect, the carboxylic acid intermediate having formula I, is added to a polar aprotic solvent (eg, acetonitrile) to form a mixture, the tetramethylchloroformamidinium hexafluorophosphate (TCFH) and the amino intermediate 46 are sequentially added, and the N,N-diisopropylethylamine (DiPEA) is added subsequently to the mixture; suitably wherein the N,N-diisopropylethylamine (DiPEA) is added subsequently to the mixture in limited amounts over a period of time, such as dropwise.

In one embodiment of the method of the first aspect, for each about 1 g of the carboxylic acid intermediate having formula I, the amino intermediate 46 and the tetramethylchloroformamidinium hexafluorophosphate (TCFH) are mixed in an amount of the polar aprotic solvent selected from the group consisting of: 1 to 100 ml, 2 to 80 ml, 3 to 60 ml, 4 to 50 ml, 10 to 50 ml, 20 to 50 ml, 20 to 40 ml, 5 to 40 ml, 8 to 30 ml, 10 to 20 ml, 12 to 18 ml, 13 to 17 ml, and 14 to 16 ml. In one particular such embodiment, for each about 1 g of the carboxylic acid intermediate having formula I, the amino intermediate 46 and the tetramethylchloroformamidinium hexafluorophosphate (TCFH) are mixed in about 15 ml of, or in about 30 ml of, the polar aprotic solvent.

In one embodiment of the method of the first aspect, the reaction is carried out in a temperature range selected from the group consisting of: −20° C. to 83° C., 0° C. to 80° C., 23° C. (room temperature) to 60° C., 30° C. to 55° C. and 35° C. to 45° C. In one particular such embodiment, the reaction is carried out at about 35° C. or 40° C., or at about 50° C.

In one embodiment of the method of the first aspect, each about 1 Equiv. of the carboxylic acid intermediate having formula I is reacted in the presence of an Equiv. amount of the N,N-diisopropylethylamine (DiPEA) selected from the group consisting of: 0.5 to 20 Equiv., 0.8 to 10 Equiv., 1 to 8 Equiv., 2 to 5 Equiv., 1 to 4 Equiv., 2.5 to 4.5 Equiv., 3 to 4 Equiv., 3 to 3.8 Equiv., 3.1 to 3.5 Equiv., and 3.2 to 3.4 Equiv., in each case of the N,N-diisopropylethylamine (DiPEA). In one particular such embodiment, each about 1 Equiv. of the carboxylic acid intermediate having formula I is reacted in the presence of between 2 and 3 Equiv. of the N,N-diisopropylethylamine (DiPEA).

In one embodiment of the method of the first aspect, the N,N-diisopropylethylamine (DiPEA) is added (eg, in limited amounts, such as dropwise) to the mixture over a period of time selected from the group consisting of: 1 min to h, 2 min to 12 h, 3 min to 8 h, 4 min to 4 h, suitably wherein the N,N-diisopropylethylamine (DiPEA) is added to the mixture over a period of time of about 5 min; or wherein the N,N-diisopropylethylamine (DiPEA) is added to the mixture over a period of time of between 1 and 2 h, such as about 1.5 h, or is added to the mixture over a period of time of about 2 h, about 3 h or about 3.5 h.

In one embodiment of the method of the first aspect, for each about 1 Equiv. of carboxylic acid intermediate having formula I, is added to between 10 and 20 L per 550 g of carboxylic acid intermediate I (such as to between 16 and 17 L per 550 g of carboxylic acid intermediate) of acetonitrile to form a mixture, and heated to reflux and concentrated (eg, between 25 and 75%, such to about 50% of the initial volume of acetonitrile). The mixture may be cooled (eg to about 20° C.) and between 1.2 to 1.6 Equiv. of (such as between 1.4 and 1.5 Equiv. of) the tetramethylchloroformamidinium hexafluorophosphate (TCFH) and between 1.1 to 1.5 Equiv. of (such as between 1.1 and 1.2 Equiv. of) the amino intermediate 46 are sequentially added. The resulting mixture may be heated to between 30° C. and 50° C. (such to about 40° C.) and between 1.5 and 6 Equiv., of (such as between 2.5 and 2.7 Equiv. of) the N,N-diisopropylethylamine (DiPEA) is added subsequently to the mixture over a period of between 30 min and 3 h (such as over a period of between 1 and 2 h); suitably wherein the N,N-diisopropylethylamine (DiPEA) is added subsequently to the mixture in limited amounts over a period of time, such as dropwise. Such heated mixture may be stirred for between 5 and 30 min (such as about 10 min) at between 30° C. and 50° C. (such as to about 40° C.) and then cooled (eg to about 20° C.). Between 5 and 20 L of (such as between 9 and 12 L of) about 0.25M NaOH may be added over a period of time (eg, about 30 min) and the mixture stirred at the (cooled) temperature for between 12 and 36 h (such as for between 20 and 24 h). Solid E9 may be recovered by filtration and, for example, dried under vacuum.

In one embodiment of the method of the first aspect, the method further comprises a step of (first) preparing the carboxylic acid intermediate having formula I:

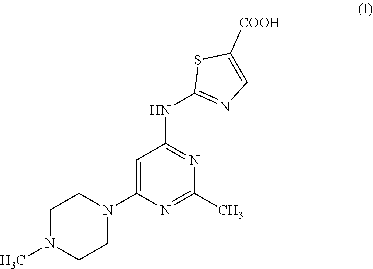 [SMILES: Cc1nc(Nc2ncc(C(=O)O)s2)cc(N2CCN(C)CC2)n1]

by hydrolysis of an ester intermediate having the formula Ia:

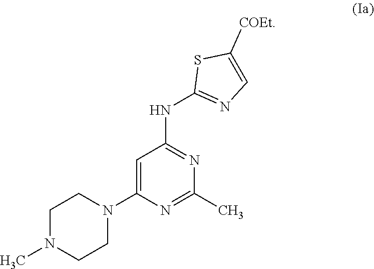

In one embodiment of the method of the first aspect, the hydrolysis of the ester intermediate having the formula Ia comprises reacting the ester intermediate having formula Ia with a hydroxide, suitably wherein the hydrolysis comprises reacting the ester intermediate having formula Ia with sodium hydroxide in a solvent mixture comprising methanol and water.

In another particular of such embodiment, the hydrolysis of the ester intermediate having formula Ia comprises reacting it with sodium hydroxide dissolved in water. In one of such particular embodiments, each about 1 Equiv. of the ester intermediate having formula Ia is reacted with between 2 and 20 Equiv (such as between 3 and 5 Equiv.) of sodium hydroxide in between 4 and 16 L per 1 kg of ester intermediate having formula Ia (such as between 7 and 8.5 L per 1 kg of ester intermediate having formula Ia) of water as a solvent, and (optionally) the mixture heated to between 5° and 70° C. (such as about 60° C.) for between 12 and 48 h (such as about 32 h). After cooling (eg to about 20° C.), the reaction may be neutralised (eg, using HCl, such as about 150 mL of about 6M HCl per 566 g of sodium hydroxide) and carboxylic acid intermediate I can be isolated as a solid by filtration, and for example, dried under vacuum. Alternatively, or additionally, before filtration, the product may be reslurried using a solvent such as acetonitrile. Purification of the product may further include one or more hot and cold cycles. For example, the mixture may be heated to about 50° C. to 70° C. (preferably to about 60° C.), is optionally stirred at about 60° C. and then cooled to about 10° C. to 30° C. (preferably to about 20° C.). The cold-hot cycle may be repeated for one, preferably two more times, and the carboxylic acid intermediate I can be isolated as a solid by filtration, and for example, dried under vacuum.

In one embodiment of the method of the first aspect, the method further comprises a step of (first) preparing the ester intermediate having formula Ia by reacting under coupling conditions compound 3

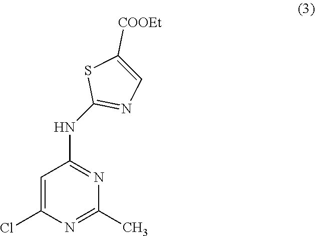 [SMILES: CCOC(=O)c1cnc(Nc2cc(Cl)nc(C)n2)s1]

with methylpiperazine.

In one embodiment of the method of the first aspect, the coupling conditions under which compound 3 is reacted with methylpiperazine in the presence of a non-nucleophilic base, such as one described above. In one particular such embodiment, the coupling conditions under which compound 3 is reacted with methylpiperazine comprise the presence of N,N-diisopropylethylamine (DiPEA). For example, each about 1 Equiv. of compound 3 may be suspended in a solvent (eg between 10 and 15 L, such as about 12 L of n-butanol, for each about 1.2 kg of compound 3) and between 1 and 3 Equiv of (such as between 1.4 and 1.6 Eqiv. of) the methylpiperazine and between 1.5 and 4 Equiv. of (such as about 2 Equiv. of) the N,N-diisopropylethylamine (DiPEA) are sequentially added, and (optionally) the reaction heated (eg heated at reflux) for between 2 and 10 hours (eg for about 5 h). After cooling (eg to 20° C.), water may be charged and the slurry aged (eg at 20° C.) for between 1 and 5 hours (eg, for about 3 h). The ester intermediate having formula Ia may be isolated as a solid by filtration, and (for example) dried under vacuum.

In one alternative embodiment of the method of the first aspect, the coupling conditions under which compound 3 is reacted with methylpiperazine may comprise a step before recovering the compound Ia in which step the mixture is subjected to at least one hot and cold cycle (preferably directly subsequent to aging). Preferably, the additional step involves heating the mixture, optionally stirring at the hot temperature, and subsequent cooling of the mixture to complete one cycle. More preferably at least two, or at least three cycles are performed. Preferably, during the hot and cold cycle the mixture is cooled to a temperature in the range of about 10° C. to about 30° C. (preferably to about 15° C. to about 25° C.; most preferably to about 20° C.). In an additionally preferred embodiment, the hot and cold cycle involves heating the mixture to about 50° C. to about 70° C., more preferably 55° C. to about 65° C., most preferably to about 60° C. Most preferably a hot and cold cycle involves cooling to about 20° C. and heating to about 60° C.

Accordingly, in the one alternative embodiment of the method of the first aspect, the coupling conditions under which compound 3 is reacted with methylpiperazine in the presence of a non-nucleophilic base, such as one described above. In one particular such embodiment, the coupling conditions under which compound 3 is reacted with methylpiperazine comprise the presence of N,N-diisopropylethylamine (DiPEA). For example, each about 1 Equiv. of compound 3 may be suspended in a solvent (eg between 10 and 15 L, such as about 12 L of n-butanol, for each about 1.2 kg of compound 3) and between 1 and 3 Equiv of (such as between 1.4 and 1.6 Eqiv. of) the methylpiperazine and between 1.5 and 4 Equiv. of (such as about 2 Equiv. of) the N,N-diisopropylethylamine (DiPEA) are sequentially added, and (optionally) the reaction heated (eg heated at reflux) for between 2 and 10 hours (eg for about 4 h). After cooling (eg to 20° C.), water may be charged and the slurry aged (eg at 20° C.) for between 1 and 5 hours (eg, for about 2 h). The mixture may then according to the alternative embodiment be heated to about 50° C. to 70° C. (preferably to about 60° C.), is optionally stirred at about 60° C. and then cooled to about 10° C. to 30° C. (preferably to about 20° C.). The cold-hot cycle may be repeated for one, preferably two more times. The ester intermediate having formula Ia may be isolated as a solid by filtration, and (for example) dried under vacuum.

In one embodiment of the method of the first aspect, compound 3 is (first) prepared by reacting under coupling conditions compound 1 with compound 2

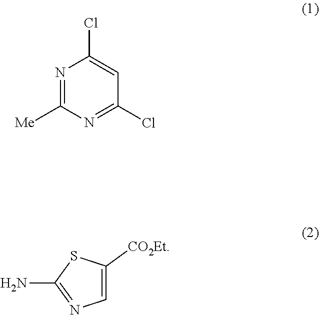 [SMILES: Cc1nc(Cl)cc(Cl)n1 ; CCOC(=O)c1cnc(N)s1]

Compound 3 (see below) is described by the present applicant in WO2020/083926 and in co-pending PCT/EP2021/060338, and compound 3 may be prepared from compounds 1 and 2 as described therein.

| | | |
|---|---|---|
| 3 | 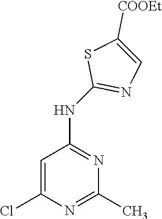 [SMILES: CCOC(=O)c1cnc(Nc2cc(Cl)nc(C)n2)s1] | ethyl 2-((6-chloro-2-methylpyrimidin- 4-yl)amino)thiazole-5-carboxylate |
| 1 | 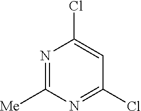 | 4,6-dichloro-2-methylpyrimidine |
| 2 | 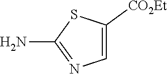 [SMILES: CCOC(=O)c1cnc(N)s1] | ethyl 2-aminothiazole-5-carboxylate |

In one embodiment of the method of the first aspect, the coupling conditions under which compound 1 and 2 are reacted comprise reacting compound 1 with compound 2 in the presence of a base in a polar aprotic solvent. For example, and as described by the present applicant in in co-pending PCT/EP2021/060338, suitable coupling conditions may comprise reacting compound 1 with compound 2 in the presence of sodium hydride in dimethylformamide (DMF), wherein the DMF may be used as a solvent.

In one particular such embodiment of the method of the first aspect, wherein the coupling conditions under which compounds 1 and 2 are reacted comprise reacting compound 1 with compound 2 in the presence of cesium carbonate in dimethylformamide (DMF), wherein the DMF may be used as a solvent (eg in an amount of about 1.5 L to 2.5 L, such as about 2 L, per 350 to 450 g (such as about 430 g) of compound 2). For example, compound 1 may be dissolved in the DMF (eg at about 20° C.) and compound 2 (about 1 Equiv.) added. The resulting mixture may be cooled to between 2 and 10° C. (eg to about 5° C.) and the cesium carbonate (about 2 1 Equiv.) added. The mixture may be warmed to between 1° and 30° C. (eg to about 20° C.) and sired for between 12 and 36 h (eg for about 22 h). The mixture may be cooled to between 5 and 20° C. (eg to about 10 C) and water added. The mixture may be warmed to between 1° and 20° C. (eg to about 20° C.) and stirred for between 10 and 20 h (eg, for about 15 h). Compound 3 may be isolated as a solid by filtration.

In one embodiment of the method of the first aspect, the amino intermediate is compound 46, and the method further comprises a step of first preparing compound 46

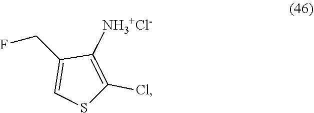

by reacting compound 45

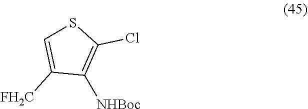

with HCl, with dioxane as solvent.

Compound 46 (see below) is described by the present applicant in co-pending PCT/EP2021/060338 (unpublished on the priority date of the present invention), and in one particular of such embodiment, the compound 46 may be prepared from compound 45 essentially as described therein.

| | | |
|---|---|---|
| 46 | 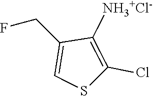 | 2-chloro-4-(fluoromethyl)thiophen-3- amine hydrochloride |
| 45 | 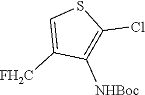 | tert-butyl (2-chloro-4- (fluoromethyl)thiophen-3-yl)carbamate |

In one alternative embodiment of the method of the first aspect, the amino intermediate is compound 46, and the method further comprises a step of (first) preparing compound 46

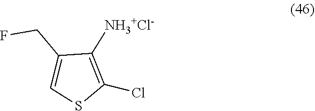

by reacting compound 45

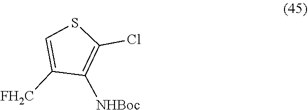

with HCl and ethyl acetate (EA) (such as with HCl, with EA as solvent).

In one embodiment of the method of the first aspect, compound 46 is prepared by reacting each about 1 Equiv. of compound 45 with between 1M and 6M HCl and between 2 and 15 Equiv. of ethyl acetate.

In one embodiment of the method of the first aspect, compound 46 is prepared by reacting each about 1 Equiv. of compound 45 with about 4M HCl and about 4 Equiv. of, or about 10 Equiv. of, ethyl acetate.

In one embodiment of the method of the first aspect, compound 46 is prepared by reacting compound 45 with HCl and ethyl acetate (EA), wherein the reaction is conducted at between 5 and 40° C. for between 1 and 24 h.

In one embodiment of the method of the first aspect, compound 46 is prepared by reacting compound 45 with HCl and ethyl acetate (EA), wherein the reaction is conducted at between 1° and 30° C. for between 4 and 18 h.

In one embodiment of the method of the first aspect, compound 46 is prepared by reacting each about 1 Equiv. of compound 45 with about 4M HCl and about 10 Equiv. of ethyl acetate and the reaction is conducted at between 2° and 30° C. for about 18 h, or by reacting each about 1 Equiv. of compound 45 with about 4M HCl and about 4 Equiv. of ethyl acetate and the reaction is conducted at between 2° and 30° C. for about 4 h.

In one embodiment of the method of the first aspect, the method further comprises a step of (first) preparing compound 45 by reacting compound 40

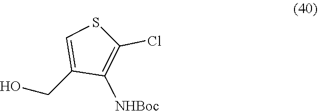 [SMILES: CC(C)(C)OC(=O)Nc1c(CO)csc1Cl]

with a fluorination agent, suitably wherein the method further comprises a step of (first) preparing compound 45 by reacting compound 40 with diethylaminosulfur trifluoride (DAST) in dichloromethane as solvent.

In one embodiment of the method of the first aspect, the method further comprises a step of (first) preparing compound 40 by reacting compound 39

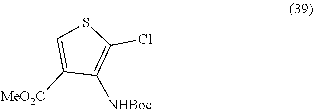 [SMILES: COC(=O)c1csc(Cl)c1NC(=O)OC(C)(C)C]

with a reducing agent, suitably wherein the method further comprises a step of (first) preparing compound 40 by reacting compound 39 with diisobutylaluminium hydride (DIBAL) in toluene as solvent.

In one embodiment of the method of the first aspect, the method further comprises a step of (first) preparing compound 39 by reacting compound 38

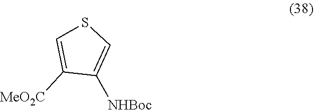 [SMILES: COC(=O)c1cscc1NC(=O)OC(C)(C)C]

with a chlorination agent, suitably wherein the method further comprises a step of (first) preparing compound 39 by reacting compound 38 with N-chlorosuccinimide (NCS) in glacial acetic acid as solvent.

In one embodiment of the method of the first aspect, the method further comprises a step of (first) preparing compound 38 by reacting compound 37

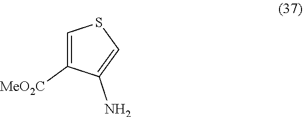

with di-tert-butyl dicarbonate, suitably wherein the method further comprises a step of (first) preparing compound 38 by reacting compound 37 with di-tert-butyl dicarbonate in dioxane as solvent.

In one embodiment of the method of the first aspect, the method further comprises a step of (first) preparing compound 37 by reacting compound 36

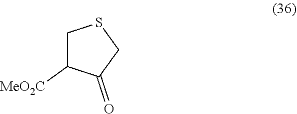

with hydroxylamine hydrochloride in acetonitrile as solvent.

In one embodiment of the method of the first aspect, the method further comprises a step of (first) preparing compound 36 by reacting compound 35

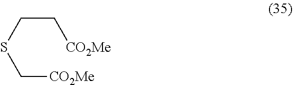 [SMILES: COC(=O)CCSCC(=O)OC]

with a base, suitably wherein the method further comprises a step of (first) preparing compound 36 compound 35 with NaH in THF as solvent.

In one embodiment of the method of the first aspect, the method further comprises a step of first preparing compound 35 by reacting compound 33 and compound 34

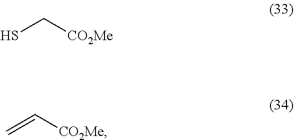

suitably the method further comprises a step of first preparing compound 35 by reacting compound 33 and compound 34 in the presence of piperidine.

In alternative embodiments of the method of the first aspect, the method further comprises a step of preparing compound 45 by a process that includes a step that comprises the applicable new reaction conditions for such step, as summarised in the respective row of Table 9.1 of Example 9. For example, the step of converting compound 39 to compound 40 may comprise conditions using LiAlH₄ with THF (eg, as solvent), the step of converting compound 38 to compound 39 may comprise conditions using NCS, and AcOH with THF (eg, as solvent), the step of converting compound 35 to compound 36 may comprise conditions using EtONa, and/or the step of reacting compounds 33 and 34 to form compound 35 may comprise conditions using Et₃N (eg, with EtOH as solvent).

In a second aspect, the present invention provides a method of preparing an amino intermediate useful for preparing the compound E9

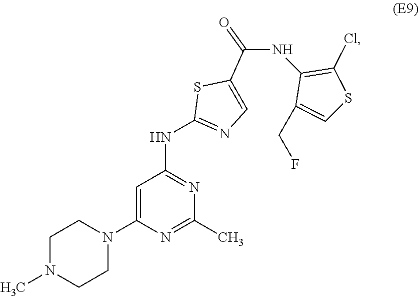 [SMILES: Cc1nc(Nc2ncc(C(=O)Nc3c(CF)csc3Cl)s2)cc(N2CCN(C)CC2)n1]

wherein the amino intermediate is 46

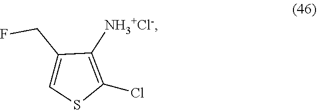

wherein the method comprises a step of reacting compound 45

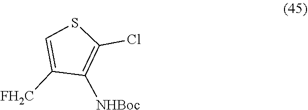

with HCl and ethyl acetate (EA) (such as with HCl, with EA as solvent).

In one embodiment of the method of the second aspect, is a method of preparing an amount of compound 46 that is a bulk amount of compound 46, such as in an amount that is greater than: about 1.5 g, about 2.5 g, about 5 g, about 15 g, about 25 g, about 40 g, or about 75 g, in particular for preparing an amount of compound 46 that is greater than about 2.5 g. For example, such method may prepare an amount of compound 46 that is greater than an amount selected from the group consisting of: about 15 g, about 20 g, about 25 g, about 40 g, about 50 g about 60 g, about 75 g, and about 80 g. Indeed, the method of the second aspect may be used to prepare greater amounts of compound 46, such as greater than about 100 g of 46, in particular, to prepare more than about 125 g, about 150 g, about 175 g or about 200 g of compound 46. In certain embodiment, the amount of compound prepared may be an amount of up to about 100 g, 200 g, 500 g or more, such as up to about 1 kg, or more, of compound 46. In one particular embodiment, the method of the second aspect is a method of preparing an amount of compound 46 that is between 2.5 g and 250 g of compound 46, such as between 4 g and 400 g of compound 46. Any of such amounts (or ranges of amounts) of compound as described in this paragraph, when applied to an amount of compound 46, can be described as a “bulk amount” of such compound.

Such an amount of compound 46 may be prepared (or contained) in one or more vessels (eg reaction vessels), containers or receptacles. In one particular such embodiment, the method of the first aspect of the invention, is used to prepare such an amount of compound 46 in a single (eg, reaction) vessel (such as a reaction flask).

In one embodiment of the method of the first aspect of the invention, the method is used to prepare a (eg, substantially) pure form (eg, as described above) of compound 46. In particular, a pure form of compound 46 may be one that contains less than 2% or 1% of total impurities (eg, as may be determined by area by HPLC).

In one embodiment of the method of the second aspect, each about 1 Equiv. of compound 45 is reacted with between 1M and 6M HCl and between 25 and 15 Equiv. of ethyl acetate.

In one embodiment of the method of the second aspect, each about 1 Equiv. of compound 45 is reacted with about 4M HCl and about 4 Equiv. of, or about 10 Equiv. of, ethyl acetate.

In one embodiment of the method of the second aspect, the reaction of compound 45 with HCl and ethyl acetate (EA) is conducted at between 5 and 40° C. for between 1 and 18 h.

In one embodiment of the method of the second aspect, the reaction of compound 45 with HCl and ethyl acetate (EA) is conducted at between 1° and 30° C. for between 4 and 18 h.

In one embodiment of the method of the second aspect, each about 1 Equiv. of compound 45 is reacted with about 4M HCl and about 10 Equiv. of ethyl acetate and the reaction is conducted at between 2° and 30° C. for about 18 h, or by reacting each about 1 Equiv. of compound 45 with about 4M HCl and about 4 Equiv. of ethyl acetate and the reaction is conducted at between 2° and 30° C. for about 4 h.

In one embodiment of the method of the second aspect, the method further comprises a step of (first) preparing compound 45 by reacting compound 40

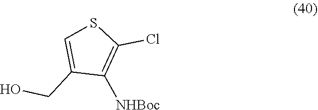 [SMILES: CC(C)(C)OC(=O)Nc1c(CO)csc1Cl]

with a fluorination agent, suitably wherein the method further comprises a step of (first) preparing compound 45 by reacting compound 40 with diethylaminosulfur trifluoride (DAST) in dichloromethane as solvent.

In one embodiment of the method of the second aspect, wherein the method further comprises a step of (first) preparing compound 40 by reacting compound 39

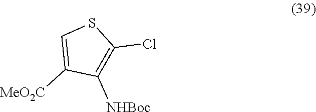 [SMILES: COC(=O)c1csc(Cl)c1NC(=O)OC(C)(C)C]

with a reducing agent, suitably wherein the method further comprises a step of (first) preparing compound 40 by reacting compound 39 with diisobutylaluminium hydride (DIBAL) in toluene as solvent.

In one embodiment of the method of the second aspect, the method further comprises a step of (first) preparing compound 39 by reacting compound 38

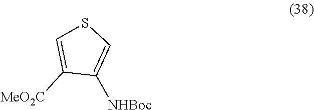 [SMILES: COC(=O)c1cscc1NC(=O)OC(C)(C)C]

with a chlorination agent, suitably wherein the method further comprises a step of (first) preparing compound 39 by reacting compound 38 with N-chlorosuccinimide (NCS) in glacial acetic acid as solvent.

In one embodiment of the method of the second aspect, wherein the method further comprises a step of (first) preparing compound 38 by reacting compound 37

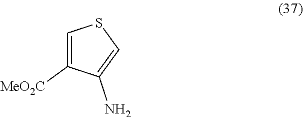

with di-tert-butyl dicarbonate, suitably wherein the method further comprises a step of (first) preparing compound 38 by reacting compound 37 with di-tert-butyl dicarbonate in dioxane as solvent.

In one embodiment of the method of the second aspect, the method further comprises a step of (first) preparing compound 37 by reacting compound 36

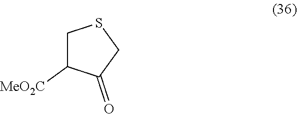

with hydroxylamine hydrochloride in acetonitrile as solvent.

In one embodiment of the method of the second aspect, the method further comprises a step of (first) preparing compound 36 by reacting compound 35

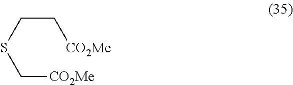 [SMILES: COC(=O)CCSCC(=O)OC]

with a base, suitably wherein the method further comprises a step of (first) preparing compound 36 by reacting compound 35 with NaH in THF as solvent.

In one embodiment of the method of the second aspect, the method further comprises a step of (first) preparing compound 35 by reacting compound 33 and compound 34

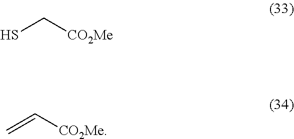

In alternative embodiments of the method of the second aspect, the method further comprises a step of preparing compound 45 by a process that includes a step that comprises the applicable new reaction conditions for such step, as summarised in the respective row of Table 9.1 of Example 9. For example, the step of converting compound 39 to compound 40 may comprise conditions using LiAlH₄ with THF (eg, as solvent), the step of converting compound 38 to compound 39 may comprise conditions using NCS, and AcOH with THF (eg, as solvent), the step of converting compound 35 to compound 36 may comprise conditions using EtONa, and/or the step of reacting compounds 33 and 34 to form compound 35 may comprise conditions using Et₃N (eg, with EtOH as solvent).

In a third aspect, the present invention provides an intermediate useful for preparing the compound E9

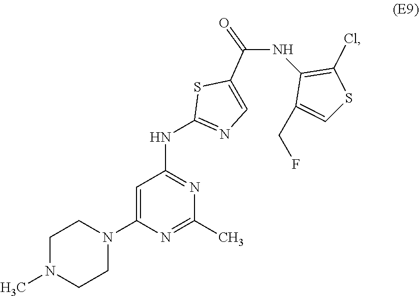 [SMILES: Cc1nc(Nc2ncc(C(=O)Nc3c(CF)csc3Cl)s2)cc(N2CCN(C)CC2)n1]

wherein the intermediate is a carboxylic acid intermediate having the formula I:

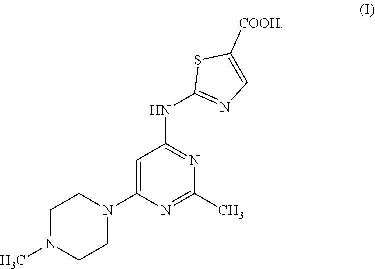 [SMILES: Cc1nc(Nc2ncc(C(=O)O)s2)cc(N2CCN(C)CC2)n1]

In a fourth aspect, the present invention provides an intermediate useful for preparing the compound E9

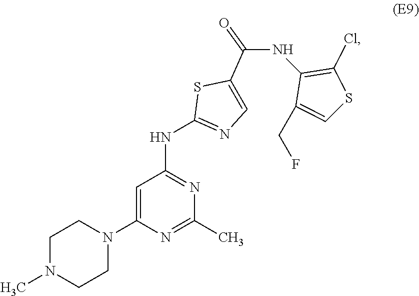 [SMILES: Cc1nc(Nc2ncc(C(=O)Nc3c(CF)csc3Cl)s2)cc(N2CCN(C)CC2)n1]

wherein the intermediate is an ester intermediate having the formula Ia:

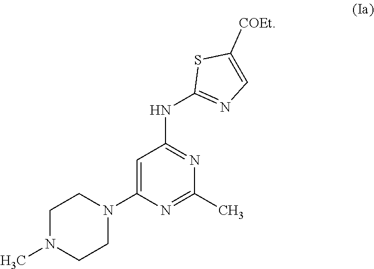

In one fifth aspect, the present application provides an amount (eg a bulk amount) of at least about 125 g of compound E9

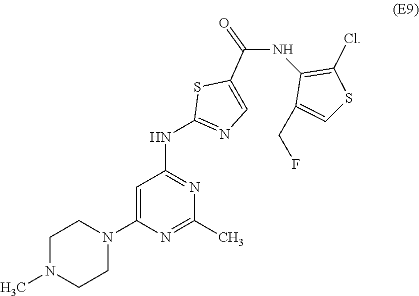 [SMILES: Cc1nc(Nc2ncc(C(=O)Nc3c(CF)csc3Cl)s2)cc(N2CCN(C)CC2)n1]

In another fifth aspect, the present application provides an amount (eg a bulk amount) of at least about 125 g of the carboxylic acid intermediate having the formula I:

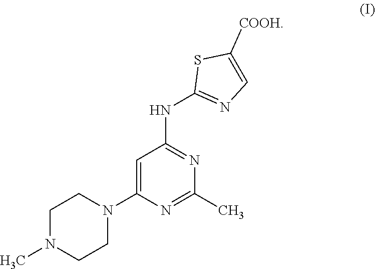 [SMILES: Cc1nc(Nc2ncc(C(=O)O)s2)cc(N2CCN(C)CC2)n1]

In a related other fifth aspect, the present application provides an amount (eg a bulk amount) of at least about 125 g of the ester intermediate having the formula Ia:

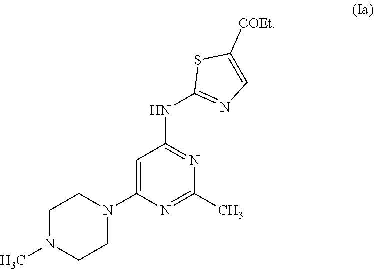

In a further fifth aspect, the present application provides an amount (eg a bulk amount) of at least about 2.5 g of the amino intermediate 46

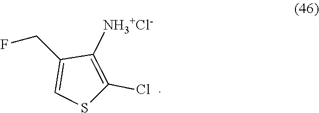

In certain embodiments of those fifth aspects of the invention relating to a bulk amount of compound E9, the carboxylic acid intermediate having formula I or the ester intermediate having formula Ia, such bulk amount can be greater than an amount selected from the group consisting of: about 150 g, about 200 g, about 250 g, about 400 g, about 600 g, about 750 g, and about 800 g. Indeed, the amount of such compound may be even greater, such as greater than about 1.0 kg of such compound, in particular, more than about 1.25 kg, about 1.5 kg, about 1.75 kg or about 2 kg of the compound. In certain embodiments, the amount of compound may be an amount of up to about 1.0 kg, 2.0 kg, 5 kg or more, such as up to about 10 kg, or more, of a compound. In one particular embodiment, the amount of compound is between 250 g and 25 kg of such compound, such as between 400 g and 4.0 kg of the compound.

In certain embodiments of those fifth aspects of the invention relating to a bulk amount of compound 46, such bulk amount can be is greater than: about 2.5 g, about 5 g, about 15 g, about 25 g, about 40 g, or about 75 g, in particular an amount of compound 46 that is greater than about 5 g. For example, such bulk amount of compound 46 is greater than an amount selected from the group consisting of: about 15 g, about 20 g, about 25 g, about 40 g, about 50 g about 60 g, about 75 g, and about 80 g. Indeed, bulk amount of compound 46 may be greater than about 100 g of 46, in particular, more than about 125 g, about 150 g, about 175 g or about 200 g of compound 46. In certain embodiment, the amount of the compound may be up to about 100 g, 200 g, 500 g or more, such as up to about 1 kg, or more, of compound 46. In one particular embodiment, the bulk amount of compound 46 is between 2.5 g and 250 g of compound 46, such as between 4 g and 400 g of compound 46.

In certain embodiments of any of the various fifth aspects of the invention, the respective amount of each compound (eg, compound E9, carboxylic acid intermediate having formula I, ester intermediate having formula Ia, or compound 46) may be present (eg, prepared, contained, transported or stored) in one of more vessels (eg a reaction vessel), containers or receptacles, that in aggregate contained the respective amount of the compound. In one particular embodiment the (bulk) amount of the applicable compound of a fifth aspect is present (eg, prepared, contained, transported or stored) in a single vessel (eg a reaction vessel), container or receptacle.

In one embodiment of the method of the first or the second aspect, the method further comprises the steps: providing the compound E9 or the amino intermediate 46 in admixture with one or more impurities; and removing at least a fraction of the impurities from the admixture.

In certain embodiments of such embodiment, suitable methods to remove a fraction of the impurities are well known and include eg column chromatography, selective precipitation, trituration and elution of impurities with a suitable solvent in which the desired compound is not soluble, etc.

The fraction of impurities removed may be such that the compound is prepared in (substantially) pure form; that is, for example, in a percentage purity as described above.

In other embodiments, the admixture is provided by synthesizing an impure form of the compound.

In particular embodiments of the any of the various fifth aspects of the invention, the (bulk) amount of the applicable compound of a fifth aspect is in (substantially) pure form; that is, for example, in a percentage purity as described above. For example, a containing less than about 2% or 1% total impurities (eg, as may be determined by area by HLPC).

In view of the above, it will be appreciated that the present invention also relates to the following itemised aspects and embodiments:
ITEM 1. A method of preparing the compound E9, especially a method of preparing greater than about 125 g of the compound E9

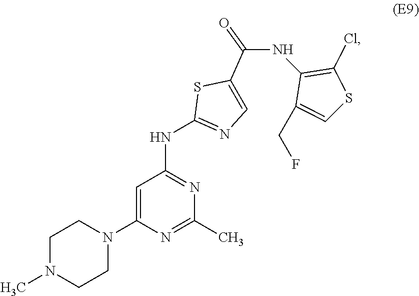 [SMILES: Cc1nc(Nc2ncc(C(=O)Nc3c(CF)csc3Cl)s2)cc(N2CCN(C)CC2)n1]

wherein the method comprises a step of reacting under coupling conditions a carboxylic acid intermediate having the formula I:

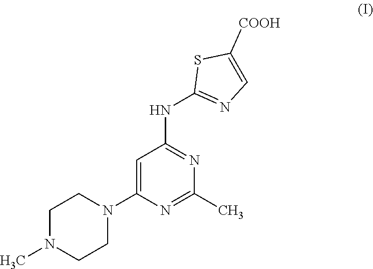 [SMILES: Cc1nc(Nc2ncc(C(=O)O)s2)cc(N2CCN(C)CC2)n1]

with an amino intermediate having the formula II:

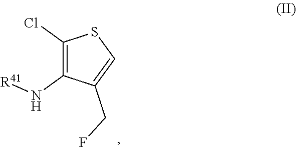

wherein R⁴¹ is selected from the group consisting of H or an amino protecting group.
ITEM 2. The method of item 1, wherein the compound E9 is prepared in a single vessel and/or is prepared in a pure form.
ITEM 3. The method of item 1 or 2, wherein R⁴¹ is an amino protecting group and the amino protecting group is selected from the group consisting of: tert-butyloxycarbonyl (BOC), 9-fluorenylmethoxycarbonyl (FMOC), benzyloxycarbonyl (Cbz), p-methoxybenzylcarbonyl (MOZ), acetyl (Ac), trifluoroacetyl, benzoyl (Bz), benzyl (Bn), p-methoxybenzyl (PMB), 3,4-dimethoxyphenyl (DMPM), p-methoxyphenyl (PMP), 2,2,2-trichloroethoxycarbonyl (Troc), triphenylmethyl (trityl; Tr), toluenesulfonyl (tosyl; Ts), para-bromophenylsulfonyl (brosyl), 4-nitrobenzenesulfonyl (nosyl), and 2-nitrophenylsulfenyl (Nps), or is selected from the group consisting of: allyloxycarbonyl (alloc), and phthalimide.
ITEM 4. The method of item 1 or 2, wherein R⁴¹ is H, especially wherein the amino intermediate having formula II is compound 46.

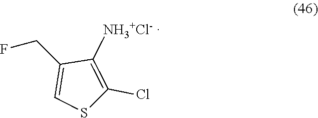

ITEM 5. The method of any of items 1 to 4, wherein the reaction of the amino intermediate II with carboxylic acid intermediate I is conducted in the presence of a coupling agent and/or a base.
ITEM 6. The method of item 5, wherein the coupling agent is selected from the group consisting of: N,N,N′,N′-tetramethylchloroformamidinium hexafluorophosphate (TCFH), 1,3-dimethyl-2-chloro-4,5-dihydro-1/fimidazolium hexafluorophosphate (DCIH) N,N,N′,N′-tetramethylfluoroformamidinium hexafluorophosphate (TFFH), 1-ethyl-3-(3-dimethylaminopropyl)carbodiimide (EDCI), dicyclohexylcarbodiimide (DCC), diisopropylcarbodiimide (DIC), 1-[bis(dimethylamino)methylene]-1H-1,2,3-triazolo[4,5-b]pyridinium 3-oxide hexafluorophosphate (HATU), 2-(1H-benzotriazol-1-yl)-1,1,3,3-tetramethyluronium hexafluorophosphate (HBTU), 2-(1H-benzotriazol-1-yl)-1,1,3,3-tetramethylaminium tetrafluoroborate (TBTU), 1-propanephosphonic anhydride (T3P), benzotriazol-1-yl-oxytripyrrolidinophosphonium hexafluorophosphate (PyBOP), 7-azabenzotriazol-1-yloxy)tripyrrolidinophosphonium hexafluorophosphate (PyAOP), and (6-chlorobenzotriazol-1-yloxy)tripyrrolidinophosphonium hexafluorophosphate (TPTDP), especially selected from the group consisting of: N,N,N′,N′-tetramethylchloroformamidinium hexafluorophosphate (TCFH), 1,3-dimethyl-2-chloro-4,5-dihydro-1H-imidazolium hexafluorophosphate (DCIH) and N,N,N′,N′-tetramethylfluoroformamidinium hexafluorophosphate (TFFH); suitably wherein the coupling agent is N,N,N′,N′-tetramethylchloroformamidinium hexafluorophosphate (TCFH).
ITEM 7. The method of item 5 or 6, wherein the base is a non-nucleophilic base, such as a non-nucleophilic base selected from the group consisting of 1-methylimidazole (NMI), 4-dimethylaminopyridine (DMAP), N,N-diisopropylethylamine (DiPEA), 2,2,6,6-tetramethylpiperidine, trimethylamine (TEA), tributylamine, 1,8-diazabicycloundec-7-ene (DBU), 1,5-diazabicyclo[4.3.0]non-5-ene (DBN), 1,5,7-triazabicyclo[4.4.0]dec-5-ene (TBD), 7-methyl-1,5,7-triazabicyclo[4.4.0]dec-5-ene (MTBD), 1,4-diazabicyclo[2.2.2]octane (TED), collidine, 1,1,3,3-tetramethylguanidine (TMG), quinuclidine, 2,2,6,6-tetramethylpiperidine (TMP), pempidine (PMP), 2,6-di-tert-butylpyridine, 2,6-lutidine, phosphazene bases (eg t-Bu-P₄), lithium diisopropylamide (LDA), sodium bis(trimethylsilyl)amide (NaHMDS), potassium bis(trimethylsilyl)amide (KHMDS), sodium tert-butoxide, and potassium tert-butoxide; especially selected from the group consisting of: 1-methylimidazole (NMI), 4-dimethylaminopyridine (DMAP), N,N-diisopropylethylamine (DiPEA) and trimethylamine (TEA); suitably wherein the base is N,N-diisopropylethylamine (DiPEA).
ITEM 8. The method of item 4, wherein the reaction comprises:
   reacting about 1 Equiv. of the carboxylic acid intermediate having formula I:

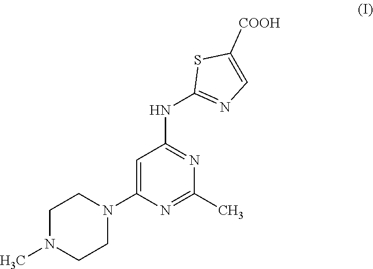 [SMILES: Cc1nc(Nc2ncc(C(=O)O)s2)cc(N2CCN(C)CC2)n1]

with between 0.5 and 5 Equiv. of the amino intermediate 46

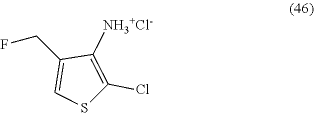

in the presence of between 0.8 and 7 Equiv. of tetramethylchloroformamidinium hexafluorophosphate (TCFH) and between 0.2 and 20 Equiv. of N,N-diisopropylethylamine (DiPEA).
ITEM 9. The method of item 8, wherein each about 1 Equiv. of the carboxylic acid intermediate having formula I is reacted with an Equiv. amount of the amino intermediate 46 selected from the group consisting of: between 1 and 4 Equiv., between 1 and 3 Equiv., between 1 and 2 Equiv. and between 1 and 1.8 Equiv., especially selected from the group consisting of: between 1 and 1.5 Equiv., between 1 and 1.4 Equiv. and between 1 and 1.3; suitably wherein each about 1 Equiv. of the carboxylic acid intermediate having formula I is reacted with between 1.1 and 1.2 Equiv. of the amino intermediate 46.
ITEM 10. The method of item 8 or 9, wherein each about 1 Equiv. of the carboxylic acid intermediate having formula I is reacted in the presence of an Equiv. amount of tetramethylchloroformamidinium hexafluorophosphate (TCFH) selected from the group consisting of: 1 to 6 Equiv., 1 to 5 Equiv., 1 to 4 Equiv., 1 to 3 Equiv., 1 to 2.5 Equiv., 1 to 2 Equiv., and 1 to 1.9 Equiv., especially selected from the group consisting of: 1.1 to 1.8 Equiv., 1.2 to 1.7 Equiv., and 1.3 to 1.6 Equiv., suitably wherein each about 1 Equiv. of the carboxylic acid intermediate having formula I is reacted in the presence of between 1.4 and 1.5 Equiv. of tetramethylchloroformamidinium hexafluorophosphate (TCFH).
ITEM 11. The method of any of items 8 to 10, wherein the carboxylic acid intermediate having formula I, the amino intermediate 46 and the tetramethylchloroformamidinium hexafluorophosphate (TCFH) are mixed in a polar aprotic solvent to form a mixture and the N,N-diisopropylethylamine (DiPEA) is added subsequently to the mixture; suitably wherein the N,N-diisopropylethylamine (DiPEA) is added subsequently to the mixture in limited amounts over a period of time, such as dropwise.
ITEM 12. The method of item 11, wherein the polar aprotic solvent is selected from the group consisting of acetone, acetonitrile, dichloromethane, dimethylformamide, dimethylpropyleneurea, dimethylsulfoxide, ethyl acetate, hexamethylphosphoric triamide, and tetrahydrofuran; suitably wherein the polar aprotic solvent is acetonitrile.
ITEM 13. The method of any of items 8 to 10, wherein the carboxylic acid intermediate having formula I, is added to a polar aprotic solvent, suitably acetonitrile, to form a mixture, the tetramethylchloroformamidinium hexafluorophosphate (TCFH) and the amino intermediate 46 are sequentially added, and the N,N-diisopropylethylamine (DiPEA) is added subsequently to the mixture; suitably wherein the N,N-diisopropylethylamine (DiPEA) is added subsequently to the mixture in limited amounts over a period of time, such as dropwise.
ITEM 14. The method of item 11, 12 or 113, wherein for each about 1 g of the carboxylic acid intermediate having formula I, the amino intermediate 46 and the tetramethylchloroformamidinium hexafluorophosphate (TCFH) are mixed in an amount of the polar aprotic solvent selected from the group consisting of: 1 to 100 ml, 2 to 80 ml, 3 to 60 ml, 4 to 50 ml, 10 to 50 ml, 20 to 50 ml, 20 to 40 ml, 5 to 40 ml, 8 to 30 ml, 10 to 20 ml, 12 to 18 ml, 13 to 17 ml, and 14 to 16 ml; suitably wherein for each about 1 g of the carboxylic acid intermediate having formula I, the amino intermediate 46 and the tetramethylchloroformamidinium hexafluorophosphate (TCFH) are mixed in about 15 ml, or in about 30 ml of, of the polar aprotic solvent.
ITEM 15. The method of any of items 8 to 14, wherein the reaction is carried out in a temperature range selected from the group consisting of: −20° C. to 83° C., 0° C. to 80° C., 23° C. (room temperature) to 60° C., 30° C. to 55° C. and 35° C. to 45°; suitably wherein the reaction is carried out at about 35° C. or 40° C., or at about 50° C.
ITEM 16. The method of any of items 8 to 15, wherein each about 1 Equiv. of the carboxylic acid intermediate having formula I is reacted in the presence of an Equiv. amount of the N,N-diisopropylethylamine (DiPEA) selected from the group consisting of: 0.5 to 20 Equiv., 0.8 to 10 Equiv., 1 to 8 Equiv., 2 to 5 Equiv., 1 to 4 Equiv., 2.5 to 4.5 Equiv., 3 to 4 Equiv., 3 to 3.8 Equiv., 3.1 to 3.5 Equiv., and 3.2 to 3.4 Equiv., in each case of the N,N-diisopropylethylamine (DiPEA); suitably wherein each about 1 Equiv. of the carboxylic acid intermediate having formula I is reacted in the presence of between 2 and 3 Equiv. of the N,N-diisopropylethylamine (DiPEA).
ITEM 17. The method of any of items 11 to 12, wherein the N,N-diisopropylethylamine (DiPEA) is added to the mixture over a period of time selected from the group consisting of: 1 min to h, 2 min to 12 h, 3 min to 8 h, 4 min to 4 h, suitably wherein the N,N-diisopropylethylamine (DiPEA) is added to the mixture over a period of time of about 5 min; or wherein the N,N-diisopropylethylamine (DiPEA) is added to the mixture over a period of time of between 1 and 2 h, such as about 1.5 h, or is added to the mixture over a period of time of about 2 h about, 3 h or about 3.5 h.
ITEM 18. The method of any of items 1 to 17, further comprising a step of preparing the carboxylic acid intermediate having formula I:

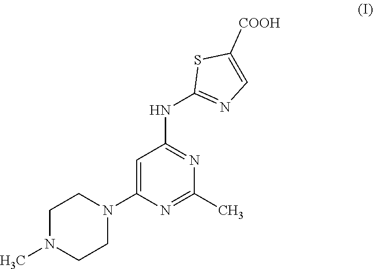 [SMILES: Cc1nc(Nc2ncc(C(=O)O)s2)cc(N2CCN(C)CC2)n1]

by hydrolysis of an ester intermediate having the formula Ia:

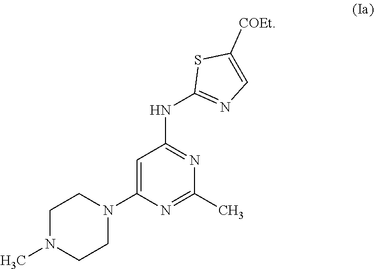

ITEM 19. The method of item 18, wherein the hydrolysis comprises reacting the ester intermediate having the formula Ia with sodium hydroxide dissolved in water.
ITEM 20. The method of item 18, wherein the hydrolysis comprises reacting the ester intermediate having formula Ia with a hydroxide, suitably wherein the hydrolysis comprises reacting the ester intermediate having formula Ia with sodium hydroxide in a solvent mixture comprising methanol and water; optionally further comprising a purification step of the product using acetonitrile; and/or comprising at least one hot and cold cycle step (before recovering the carboxylic acid of formula I), wherein the hot and cold cycle comprises heating the reaction mixture to about 55° C. to about 65° C. (preferably to about 60° C.), and a cooling the reaction mixture to about 15° C. to about 20° C. (preferably to about 20° C.
ITEM 21. The method of any of items 18 to 20, further comprising a step of preparing the ester intermediate having formula Ia by reacting under coupling conditions compound 3

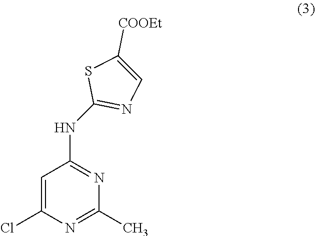 [SMILES: CCOC(=O)c1cnc(Nc2cc(Cl)nc(C)n2)s1]

with methylpiperazine.
ITEM 22. The method of item 21, wherein
   (i) the coupling conditions comprise reacting the compound 3, optionally dissolved in n-butanol as a solvent, with methylpiperazine in the presence of N,N-diisopropylethylamine (DiPEA); and/or
   (ii) the reaction mixture is subjected to at least one hot and cold cycle, wherein the hot and cold cycle comprises a heating of the mixture to about 55° C. to about 65° C. (preferably to about 60° C.), and a cooling of the mixture to about 15° C. to about 20° C. (preferably to about 20° C.).
ITEM 23. The method of item 21 or 22, wherein compound 3 is prepared by reacting under coupling conditions compound 1 with compound 2.

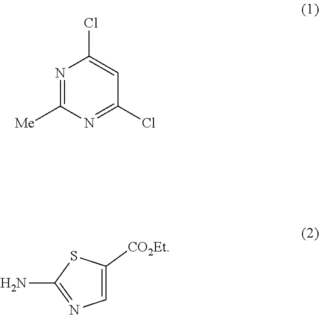 [SMILES: Cc1nc(Cl)cc(Cl)n1 ; CCOC(=O)c1cnc(N)s1]

ITEM 24. The method of item 23, wherein the coupling conditions comprise reacting compound 1 with compound 2 in the presence of a base in a polar aprotic solvent; suitably wherein the coupling conditions comprise reacting compound 1 with compound 2, in dimethylformamide (DMF), in the presence of sodium hydride.
ITEM 25. The method of item 23, wherein the coupling conditions comprise reacting compound 1 (optionally dissolved in DMF as a solvent) and compound 2 in the presence of cesium carbonate.
ITEM 26. The method of any of items 1, 2 and 4 to 25, wherein the amino intermediate is compound 46, and the method further comprises a step of preparing compound 46

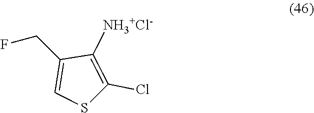

by reacting compound 45

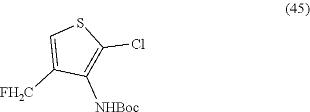

with HCl, with dioxane as solvent.
ITEM 27. The method of any of items 1, 2 and 4 to 25, wherein the amino intermediate is compound 46, and the method further comprises a step of preparing compound 46

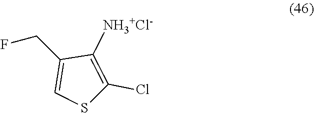

by reacting compound 45

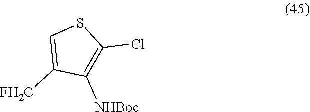

with HCl and ethyl acetate (EA).
ITEM 28. The method of item 27, wherein each about 1 Equiv. of compound 45 is reacted with between 1M and 6M HCl and between 2 and 15 Equiv. of ethyl acetate.
ITEM 29. The method of item 27 or 28, wherein each about 1 Equiv. of compound 45 is reacted with about 4M HCl and about 4 Equiv. of, or about 10 Equiv. of, ethyl acetate.
ITEM 30. The method of any of items 27 to 29, wherein the reaction is conducted at between 5 and 40° C. for between 1 and 24 h.
ITEM 31. The method of any of items 27 to 30, wherein the reaction is conducted at between 1° and 30° C. for between 4 and 18 h.
ITEM 32. The method of any of items 27 to 31, wherein about 10 Equiv. of compound 45 is reacted with about 4M HCl and about 10 Equiv. of ethyl acetate and the reaction is conducted at between 2° and 30° C. for about 18 h, or by reacting each about 1 Equiv. of compound 45 with about 4M HCl and about 4 Equiv. of ethyl acetate and the reaction is conducted at between 2° and 30° C. for about 4 h.
ITEM 33. The method of any of items 26 to 32, wherein the method further comprises a step of preparing compound 45 by reacting compound 40

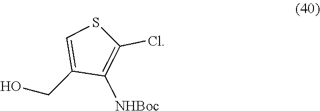 [SMILES: CC(C)(C)OC(=O)Nc1c(CO)csc1Cl]

with a fluorination agent, suitably wherein the method further comprises a step of preparing compound 45 by reacting compound 40 with diethylaminosulfur trifluoride (DAST) in dichloromethane as solvent.
ITEM 34. The method of item 33, wherein the method further comprises a step of preparing compound 40 by reacting compound 39

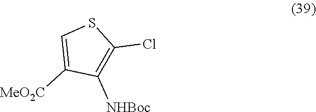 [SMILES: COC(=O)c1csc(Cl)c1NC(=O)OC(C)(C)C]

with a reducing agent, suitably wherein the method further comprises a step of preparing compound 40 by reacting compound 39 with diisobutylaluminium hydride (DIBAL) in toluene as solvent.
ITEM 35. The method of item 34, wherein the method further comprises a step of preparing compound 39 by reacting compound 38

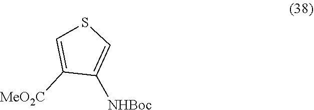 [SMILES: COC(=O)c1cscc1NC(=O)OC(C)(C)C]

with a chlorination agent, suitably wherein the method further comprises a step of preparing compound 39 by reacting compound 38 with N-chlorosuccinimide (NCS) in glacial acetic acid as solvent.
ITEM 36. The method of item 35, wherein the method further comprises a step of preparing compound 38 by reacting compound 37

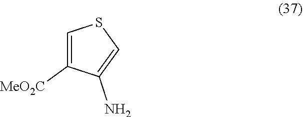

with di-tert-butyl dicarbonate, suitably wherein the method further comprises a step of preparing compound 38 by reacting compound 37 with di-tert-butyl dicarbonate in dioxane as solvent.
ITEM 37. The method of item 36, wherein the method further comprises a step of preparing compound 37 by reacting compound 36

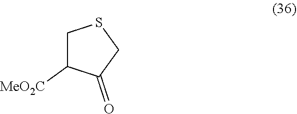

with hydroxylamine hydrochloride in acetonitrile as solvent.
ITEM 38. The method of item 37, wherein the method further comprises a step of preparing compound 36 by reacting compound 35

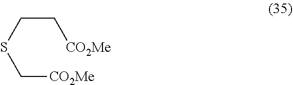 [SMILES: COC(=O)CCSCC(=O)OC]

with a base, suitably wherein the method further comprises a step of preparing compound 36 compound 35 with NaH in THF as solvent.
ITEM 39. The method of item 38, wherein the method further comprises a step of preparing compound 35 by reacting compound 33 and compound 34

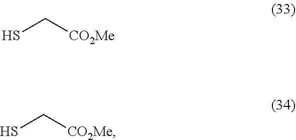 [SMILES: COC(=O)CS ; COC(=O)CS]

suitably the method further comprises a step of preparing compound 35 by reacting compound 33 and compound 34 in the presence of piperidine.
ITEM 39a. The method of any of items 26 to 32, wherein the method further comprises a step of preparing compound 45 by a process that includes a step that comprises the applicable new reaction conditions for such step, as summarised in the respective row of Table 9.1 of Example 9.
ITEM 40. A method of preparing an amino intermediate useful for preparing the compound E9

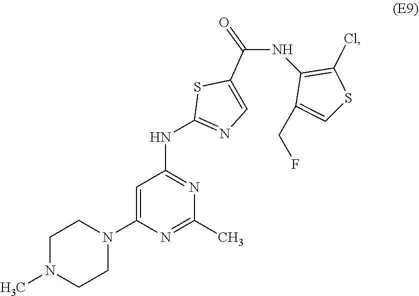 [SMILES: Cc1nc(Nc2ncc(C(=O)Nc3c(CF)csc3Cl)s2)cc(N2CCN(C)CC2)n1]

wherein the amino intermediate is 46

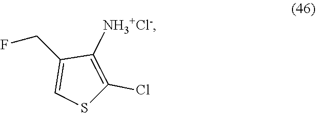

especially a method of preparing greater than about 2.5 g of the amino intermediate 46, wherein the method comprises a step of reacting compound 45

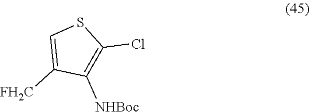

with HCl and ethyl acetate (EA).
ITEM 41. The method of item 40, wherein the amino intermediate 46, is prepared in a single vessel and/or is prepared in a pure form.
ITEM 42. The method of item 40 or 41, wherein each about 1 Equiv. of compound 45 is reacted with between 1M and 6M HCl and between 2 and 15 Equiv. of ethyl acetate.
ITEM 43. The method of item 40, 41 or 42, wherein each about 1 Equiv. of compound 45 is reacted about 4M HCl and about 4 Equiv. of, or about 10 Equiv. of, ethyl acetate.
ITEM 44. The method of any of items 40 to 43, wherein the reaction is conducted at between 5 and 40° C. for between 1 and 18 h.
ITEM 45. The method of any of items 40 to 44, wherein the reaction is conducted at between 1° and 30° C. for between 4 and 18 h.
ITEM 46. The method of any of items 40 to 45, wherein each about 1 Equiv. of compound 45 is reacted with about 4M HCl and about 10 Equiv. of ethyl acetate and the reaction is conducted at between 2° and 30° C. for about 18 h, or by reacting each about 1 Equiv. of compound 45 with about 4M HCl and about 4 Equiv. of ethyl acetate and the reaction is conducted at between 2° and 30° C. for about 4 h.
ITEM 47. The method of any of items 38 to 46, wherein the method further comprises a step of preparing compound 45 by reacting compound 40

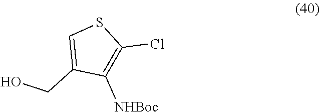 [SMILES: CC(C)(C)OC(=O)Nc1c(CO)csc1Cl]

with a fluorination agent, suitably wherein the method further comprises a step of preparing compound 45 by reacting compound 40 with diethylaminosulfur trifluoride (DAST) in dichloromethane as solvent.
ITEM 48. The method of item 47, wherein the method further comprises a step of preparing compound 40 by reacting compound 39

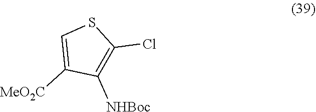 [SMILES: COC(=O)c1csc(Cl)c1NC(=O)OC(C)(C)C]

with a reducing agent, suitably wherein the method further comprises a step of preparing compound 40 by reacting compound 39 with diisobutylaluminium hydride (DIBAL) in toluene as solvent.
ITEM 49. The method of item 48, wherein the method further comprises a step of preparing compound 39 by reacting compound 38

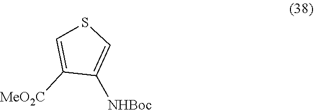 [SMILES: COC(=O)c1cscc1NC(=O)OC(C)(C)C]

with a chlorination agent, suitably wherein the method further comprises a step of preparing compound 39 by reacting compound 38 with N-chlorosuccinimide (NCS) in glacial acetic acid as solvent.
ITEM 50. The method of item 49, wherein the method further comprises a step of preparing compound 38 by reacting compound 37

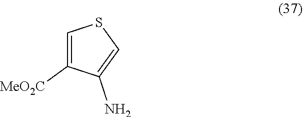

with di-tert-butyl dicarbonate, suitably wherein the method further comprises a step of preparing compound 38 by reacting compound 37 with di-tert-butyl dicarbonate in dioxane as solvent.
ITEM 51. The method of item 50, wherein the method further comprises a step of preparing compound 37 by reacting compound 36

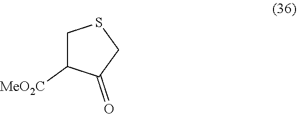

with hydroxylamine hydrochloride in acetonitrile as solvent.
ITEM 52. The method of item 51, wherein the method further comprises a step of preparing compound 36 by reacting compound 35

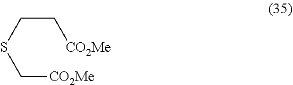 [SMILES: COC(=O)CCSCC(=O)OC]

with a base, suitably wherein the method further comprises a step of preparing compound 36 by reacting compound 35 with NaH in THF as solvent.
ITEM 53. The method of item 52, wherein the method further comprises a step of first preparing compound 35 by reacting compound 33 and compound 34

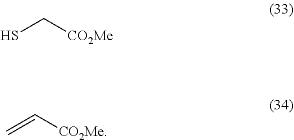

ITEM 53a. The method of any of items 38 to 46, wherein the method further comprises a step of preparing compound 45 by a process that includes a step that comprises the applicable new reaction conditions for such step, as summarised in the respective row of Table 9.1 of Example 9.
ITEM 54. An intermediate useful for preparing the compound E9

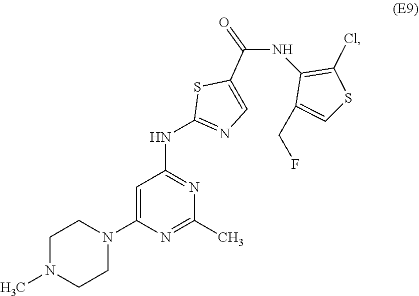 [SMILES: Cc1nc(Nc2ncc(C(=O)Nc3c(CF)csc3Cl)s2)cc(N2CCN(C)CC2)n1]

wherein the intermediate is a carboxylic acid intermediate having the formula I:

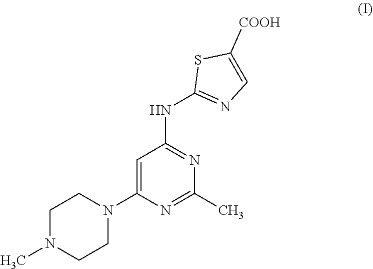 [SMILES: Cc1nc(Nc2ncc(C(=O)O)s2)cc(N2CCN(C)CC2)n1]

ITEM 55. An intermediate useful for preparing the compound E9

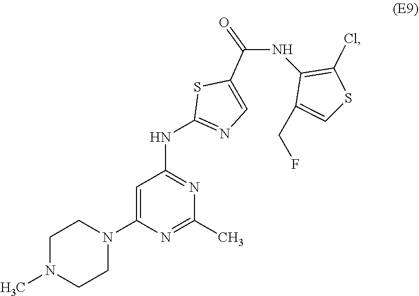 [SMILES: Cc1nc(Nc2ncc(C(=O)Nc3c(CF)csc3Cl)s2)cc(N2CCN(C)CC2)n1]

wherein the intermediate is an ester intermediate having the formula Ia:

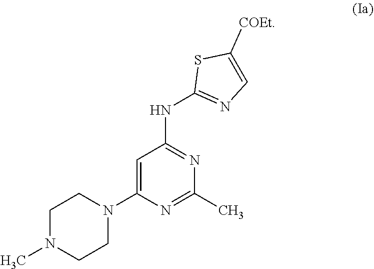

ITEM 56. A bulk amount of a compound, wherein the compound is one and, in the respective amount, selected from the group consisting of:
   (A) at least about 125 g of compound E9

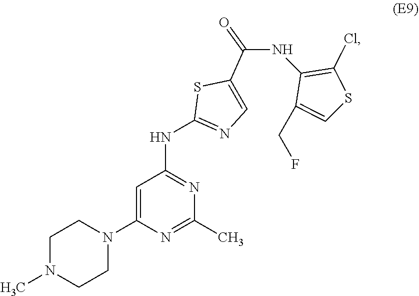 [SMILES: Cc1nc(Nc2ncc(C(=O)Nc3c(CF)csc3Cl)s2)cc(N2CCN(C)CC2)n1]

(B) at least about 125 g of the carboxylic acid intermediate having the formula I:

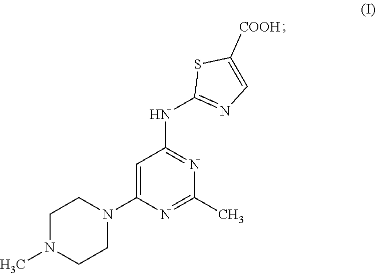 [SMILES: Cc1nc(Nc2ncc(C(=O)O)s2)cc(N2CCN(C)CC2)n1]

(C) at least about 125 g of the ester intermediate having the formula Ia:

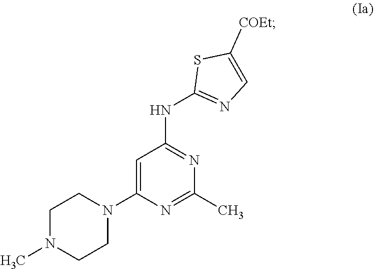

(D) at least about 2.5 g of the amino intermediate 46

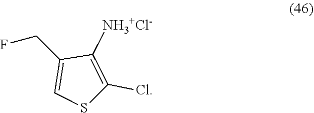

ITEM 57. The bulk amount of compound of item 56, wherein the amount of compound is present in a single vessel, container or receptacle.
ITEM 58. The bulk amount of compound of item 56 or 57, wherein compound is present in pure form.
ITEM 59. The bulk amount of compound of any of items 56 to 58 when the compound is compound E9, the carboxylic acid intermediate having the formula I or the ester intermediate having the formula Ia, wherein the bulk amount of such compound is greater than an amount selected from the group consisting of: about 200 g, about 250 g, about 400 g, about 600 g, about 750 g, and about 800 g.
ITEM 59. The bulk amount of compound of any of items 56 to 58 when the compound is compound 46, wherein the bulk amount of such compound is greater than an amount selected from the group consisting of: about 2.5 g, about 5 g, about 15 g, about 25 g, about 40 g, or about 75 g.

The examples show:

### Example 1 [Comparative]: Compound E9 is a Potent and Preferential Inhibitor of SIK3

As described by the present applicant in PCT/EP2021/060338 (unpublished on the priority date of the present invention), compound E9 (FIG. 1) was surprisingly found to inhibit SIK3 more potent inhibitor of SIK3 than the structurally similar multi kinase-inhibiting approved drug dasatinib (A8), and even a more potent inhibitor of SIK3 than the closely related published compounds B3 (WO2018/193084), C7 and D9 (both, WO2020/083926) (FIG. 1). An even more surprising property of compound E9 described by the present applicant in PCT/EP2021/060338 (unpublished) was that it was shown to be a more preferentially inhibitors towards SIK3—compared to the very closely kinase family member SIK2—than any of the other prior art compounds A8, B3, C7 or D9 (Table 1.1, data taken from applicable rows of Tables 2.1 and 3A1 of PCT/EP2021/060338).

Very surprisingly, the potency of the fluorinated-thiophene compound E9 against the target kinase SIK3 was 2-3 times improved over the other two thiophene (non-fluorinated) compounds C7 and, especially, D9.

TABLE 1.1
Biological activity of various compounds to inhibit SIK2 and SIJ3
(IC5). Compound E9 is a potent and preferential inhibitor of SIK3
compared to other structurally related prior art compounds

Kinase: IC50 (nM)

| Compound | SIK2 | SIK3 |
|---|---|---|
| A8 | <20 | 20-50 |
| B3 | 50-100 | 200-500 |
| C7 | <20 | 20-50 |
| D9 | ~10 to 15 | ~60 |
| E9 | <10 | <20 |

The IC50 of compounds A8, B3, C7, D9 and E9 were determined using standard procedures (ProQuinase, Freiburg, Germany). Briefly, and describing exemplary such procedures, a radiometric protein kinase assay (33PanQinase® Activity Assay) was used for measuring the kinase activity of the five protein kinases. All kinase assays were performed in 96-well FlashPlates™ from PerkinElmer (Boston, MA, USA) in a 50 uL reaction volume. The reaction cocktail was pipetted in four steps in the following order:
25 μL of assay buffer (standard buffer/[gamma-33P]-ATP)
10 μL of ATP solution (in water)
5 μL of test compound (in 10% DMSO)
20 uL enzyme/substrate mix

The assay for all protein kinases contained 70 mM HEPES-NaOH pH7.5, 3 mM MgCl₂, 3 mM MnCl₂, 3 μM Na-orthovanadate, 1.2 mM DTT, ATP (variable concentrations, corresponding to the apparent ATP-Km of the respective kinase, see Table 1.2), [gamma-33P]-ATP (approx. 8×10⁵ cpm per well), protein kinase (variable amount, see Table 1.2), and substrate (see Table 1.2).

The following amounts of enzyme and applicable substrate were used per well:

TABLE 1.2
Assay parameters for the tested protein kinases.

| | Kinase | Kinase | ATP | | Substrate |
|---|---|---|---|---|---|
| Kinase | Conc. | Conc. | Conc. | | Conc. |
| Name | (ng/50 μL) | (nM*) | (μM) | Substrate | (μg/50 μL) |
| SIK2 | 3 | 1 | 1.0 | RBER-CHKtide | 2 |
| SIK3 | 50 | 15.9 | 1.0 | RBER-CHKtide | 2 |

*Maximal molar enzyme assay concentrations, implying enzyme preparations exclusively containing 100% active enzyme

The reaction cocktails were incubated at 30° C. for 60 minutes. The reaction was stopped with 50 μL of 2% (v/v) H3PO4, plates were aspirated and washed two times with 200 μL 0.9% (w/v) NaCl. Incorporation of 33Pi was determined with a microplate scintillation counter (Microbeta, Wallac). All assays were performed with a BeckmanCoulter/SAGIAN™ Core System.

Compounds for this, and the other comparative examples, were synthesised as described in Example 6.

### Example 2 [Comparative]: Increased Inhibition of NFKB Activity and HDAC4 Phosphorylation by SIK3 Kinase Inhibitors, Especially by E9

The applicant described in PCT/EP2021/060338 (unpublished on the priority date of the present invention) that compound E9, showed increased inhibition of phosphorylation of HDAC4, the direct substrate of SIK3 kinase and the key mediator of NFkB activity.

Compound E9 showed enhanced biochemical inhibition of SIK3 kinase (Example 1) resulting in more potent inhibition of TNF-induced phosphorylation of HDAC4 and NFkB activity in diverse cells than the structurally similar thiophene compounds C7 and D9 as well as structurally related B3 and dasatanib (A8) (Table 2.1, taken from the applicable rows of Tables 9.1C and 9C of PCT/EP2021/060338). TNF-induced NFKB activity in MC38 cells was measured analogously to the assay in PANC-1 cells described in connection with Example 9.2 (Table 9C) of WO2020/083926), except that 10 ng/ml rMuTNF was used.

TABLE 2.1
Increased inhibition of NFKB activity and HDAC4
phosphorylation by E9 compared to other SIK3

| | Inhibition | Inhibition | Inhibition |
|---|---|---|---|
| | of NFKB | of NFKB | of HDAC4 |
| Compound | activity (MC38) | activity (PANC-1) | phosphorylation |
| A8 | + | + | ++ |
| C7 | + | + | + |
| E9 | ++ | ++ | ++ |
| D9 | NT | + | + |

+ = Significant inhibition;
++ = Strong and significant inhibition

### Example 3 [Comparative]: Improved ADMET Properties of Compound E9 Compared to Other Kinase Inhibitors Disclosed Herein

Using assays such as those described elsewhere (for example in connection with the data presented in Table 5A of WO2020/083926)), the present applicant had previously demonstrated that the (fluorinated C7-like compound) E9 showed comparable, and in some aspects even improved, drug-like properties in a number of in-vitro ADMET assays, including plasma-protein binding, stability, and cell permeation, (Table 3.1 and Table 3.2).

The applicant described showed that the fluorinated C7-like compound E9 exhibited high human and mouse plasma protein binding (>95%). However, C7 and all fluorinated C7-like compounds, as well as dasatinib (A8), showed excellent plasma stability in human and mouse plasma with T½>150 min.

Accordingly, fluorinated C7-like E9 was unstable in human and mouse liver microsomes (half-life of about 10 min, intrinsic clearance between 130-150 μL/min/mg; Table 3.1). Those microsomal stabilities classified the compounds as high clearance drugs.

Indeed, in the presence of human hepatocytes, E9 exhibited a half-life of about 25 min, therefore confirming it to be high clearance drugs. However, C7 displayed higher stability with half-life of approx. 1 h, classifying it rather as medium-to-high clearance drugs (Table 3.1).

Fluorinated C7-like derivative E9 showed improved stability in the presence of mouse hepatocytes and can be classified as a medium clearance drug, with a typical half-life of about 65 min, more stable in such model than compound C7 (Table 3.1).

TABLE 3.1
Comparable stability and solubility of
kinase inhibitors of E9 compared to C7.

Compound

| Assay | Parameter | A8 | B3 | C7 | E9 |
|---|---|---|---|---|---|
| Human | Half-life (min) | 10 | 58 | 10 | 11 |
| Liver | Intrinsic | 141 | 24 | 142 | 131 |
| microsomal | clearance | | | | |
| stability | (uL/min/mg) | | | | |
| Mouse liver | Half-life (min) | 18 | 62 | 13 | 9 |
| microsomal | Intrinsic | 72 | 22 | 110 | 158 |
| stability | clearance | | | | |
| | (uL/min/mg) | | | | |
| Human | Half-life (min) | NT | NT | 54 | 24 |
| hepatocyte | Intrinsic | NT | NT | 26 | 58 |
| stability | clearance | | | | |
| | (uL/min/mg) | | | | |
| Mouse | Half-life (min) | NT | NT | 50 | 65 |
| hepatocyte | Intrinsic | NT | NT | 28 | 21 |
| stability | clearance | | | | |
| | (uL/min/mg) | | | | |
| Human | Percentage | 2 | 7 | 2 | 1 |
| plasma- | unbound (%) | | | | |
| protein | Recovery at | NT | NT | 75 | 92 |
| binding | 4 h (%) | | | | |
| Murine | Percentage | 3 | 11 | 3 | 1 |
| plasma | unbound (%) | | | | |
| protein | Recovery at | 93 | NT | 87 | 88 |
| binding | 4 h (%) | | | | |

NT = not tested

By testing in the same, similar or analogous assays for MDR1-MDCK efflux ratio as described elsewhere (eg, in connection with the data show in Table 5D of WO2020/083926)), the applicant had showed that the efflux ratios (MDCK-wild type) of kinase inhibitors E9 (a fluorinated C7-like derivative) was in the range of 20-30 and for C7 in the range of 40-50. As these compounds exhibited effective MDCK-MDR1 efflux ratios of >10, they are most likely P-gp substrates (Table 3.2).

TABLE 3.2
Mean efflux ratios of C7 and kinase inhibitor
E9 in MDCK wild type and MDCK-MDR1 cells.

| | | Compound | |
|---|---|---|---|
| Assay | Parameter | C7 | E9 |
| MDCK-wild type cells | Mean Papp A > B (10⁻⁶ cm/s) | 9 | 10 |
| | Mean Efflux Ratio (wt) | 4.0 | 1.0 |
| MDCK-MDR1 cells | Mean Papp A > B (10⁻⁶ cm/s) | 2 | 5 |
| | Mean Efflux Ratio (MDR1) | 52 | 26 |

| | | |
|---|---|---|
| P-gP driven efflux ratio (MDR1/wt) | 13 | 27 |

### Example 4 [Comparative]: Improved Pharmacokinetic and Tolerability Properties of Compound E9 Compared to Other Kinase Inhibitors Disclosed Herein

Following an oral dose of 30 mg/Kg (po, per os), the applicant has previously demonstrated in PCT/EP2021/060338 (unpublished on the priority date of the present invention), a surprisingly improved exposure of compound E9 in mice compared to the structurally related kinase inhibitors C7, B3 and dasatinib (A8). Compound E9, was described to show reduced drug clearance and therefore PK properties superior to those of the thiophene-containing compound C7 (Table 4.1, taken from Tables 5.2C and 5.2B of PCT/EP2021/060338), and in particular, E9 was described to show a 4.5-fold and 2-fold improvement for total and free serum concentrations over C7, respectively.

TABLE 4.1
Screening PK properties of Compound E9 in mice compared
to certain other kinase inhibitors, including the
prior-art thiophene-containing compound C7

| | Plasma concentration at timepoint after 30 mg/kg po | | |
|---|---|---|---|
| | Plasma | Free plasma | |
| | concentration | concentration | Cₘₐₓ |
| Compounds | after 6 h (nM) | after 6 h (nM) | after 2 h (ng/ml) |
| A8 | 243 | 6 | 550 |
| B3 | 29 | 3 | 56 |
| C7 | 69 | 3.7 | 347 |
| D9 | NT | 8 | ND |
| E9 | 309 | 7.7 | 426 |

To further investigate these advantageous PK properties of compound E9 (fluorinated C7-like variant), it was tested in a full pharmacokinetic study with oral administration (peros, po).

The experimental procedure and the determination of the pharmacokinetic parameters was done using assays as described in connection with the data presented in Table 5.2A of WO2020/083926)).

The applicant described in PCT/EP2021/060338 the particularly surprising property of improved in vivo drug exposure and potency (Table 4.2) for compound E9 when administered orally in mice.

Surprisingly indeed, the applicant described that the Cmax for free compounds E9 was significantly higher than those for dasatinib (A8), as well especially higher than the prior art heterocycle compound B3, as well as for the structural related compound C7. Further surprisingly, the PK parameters of the tested compounds showed that compound E9 exhibited a shorter half-life to C7, but with a similar Cmax for free compound in plasma (FIG. 3, taken from FIG. 24 of PCT/EP2021/060338). These different shapes of PK curves provide opportunities for different dosage and treatment regimens to be developed for compound E9 compared to C7 as drug candidates having otherwise the same mechanism of action.

In particular, a significantly improved AUC of total and free plasma levels E9 was exhibited compared to C7, upon oral administration of these compounds. However, the differences in the free plasma levels are not as prominent as in the total plasma levels (Table 4.2), because the plasma protein binding is higher for E9 (see Table 3.1). Additionally, absorption and/or clearance of E9 was different compared to C7. In general, the applicant described the surprising observation that the fluorinated compound E9 displayed significantly improved DMPK properties compared to C7.

TABLE 4.2
DMPK properties of kinase inhibitor E9 in comparison to C7, A8 (dasatinib) and B3

| | DMPK Parameter | | | | | | | |
|---|---|---|---|---|---|---|---|---|
| | Compound | | | | | | | |
| | E9 | | C7 | | A8 (dasa) | | B3 | |
| | Dose: | | | | | | | |
| | 30 mg/kg po | | 30 mg/kg po | | 30 mg/kg po | | 30 mg/kg po | |
| | Plasma Concentration | | | | | | | |
| | Total | Free | Total | Free | Total | Free | Total | Free |
| Cₘₐₓ (ng/ml): | 2,688.72 | 66.92 | 474.16 | 21.34 | 550 | 14.4 | 99 | 10.7 |
| Tₘₐₓ (h): | 0.25 | 0.25 | 2.00 | 2.00 | 2.00 | 2.00 | 0.5 | 0.5 |
| t_{1/2} (h): | 2.76 | 2.76 | 1.42 | 1.42 | 3.0 | 3.0 | 2.4 | 2.4 |
| AUCₗₐₛₜ (nM*h): | 9,584.81 | 238.75 | 1,157.10 | 52.07 | 6,465 | NC | 589 | NC |
| AUC_{inf} (nM*h): | 9,631.95 | 239.92 | 1,306.48 | 58.79 | 6,487 | NC | 697 | NC |
| % AUCₑₓₜᵣₐₚ: | 0.49 | 0.49 | 11.24 | 11.24 | 0.3 | NC | 14 | NC |
| MRTₗₐₛₜ (h): | 3.38 | 3.38 | 2.75 | 2.75 | NC | NC | NC | NC |
| Rsq | 0.84 | 0.84 | 0.99 | 0.99 | NC | NC | NC | NC |

NC = not calculated

Taking into consideration biochemical and key pharmacokinetic properties, it can be clearly seen from Table 4.3 that compound E9 exhibits superior drug-like properties in comparison to the structurally similar thiophene compound C7, which (by comparison to the above) is itself superior to the prior art heterocyclic compound B3. Indeed, compound E9 is surprisingly potent as a SIK3 inhibitor, and shows surprisingly superior PK properties compared to the other tested compounds (and also to dasatinib, AB)

TABLE 4.3
Relative superiority of drug-like properties
for compound E9 over compound C7

| Parameter | C7 | E9 |
|---|---|---|
| SIK3 IC50 (nM) | 1x | 0.3x |
| Inhibition of NFKB activity | + | ++ |
| Inhibition of HDAC phosphorylation | + | ++ |

Total plasma concentration at 30 mg/kg p.o dose

| | | |
|---|---|---|
| C max (ng/ml) | 1x | 5.6x |
| t_{1/2} (h) | 1x | 2x |
| AUC₀₋ₗₐₛₜ (nM*h) | 1x | 8.2x |
| MRT₀₋ₗₐₛₜ (h) | 1x | 1.2x |

### Example 5 [Comparative]: In-Vivo Anti-Cancer (Solid Tumour) Efficacy of Kinase Inhibitors E9 and C7

Using assays such as those described elsewhere (such as described in Example 8 of WO2020/083926)), the applicant has described in PCT/EP2021/060338 (unpublished on the priority date of the present invention) that the kinase inhibitors E9 (a fluorinated C7-like variant) showed surprisingly significant in-vivo anti-cancer efficacy.

Similar as described in Table 8A of WO2020/083926)) (see Example 8 thereof) the following study outline was used (Table 5.1). Dosage and administration regimens for the tested kinase inhibitor E9 was adapted according to its respective SIK3 kinase inhibition and DMPK properties (see previous examples), such that its plasma free-drug concentration in relation to its SIK3 IC50 value was comparable to that for C7. Other compounds described in PCT/EP2021/060338 were analogously included in the same in study (compounds E4 and E10).

TABLE 5.1
Example treatment groups.

| | | Single dose | | | | Cumulated | | Number |
|---|---|---|---|---|---|---|---|---|
| Group | Treatment | (mg/kg) | | Dosing | Route | doses | Vehicle | of mice |
| 1 | Rat IgG2a isotyp | 10 | mg/kg | 3q7d | i.p. | 6 | Vehicle | 6 |
| | control (clone 2A3) | | | | | | | |
| 2 | Anti-mPD-1 | 10 | mg/kg | 3q7d | i.p. | 6 | Vehicle | 6 |
| | (clone RMP1-14) | | | | | | | |
| 3 | Vehicle* | 10 | mg/kg | BIDx14 | po | 28 | — | 6 |
| 4 | C7 | 100 | mg/kg | BIDx14 | po | 28 | Vehicle | 6 |
| 5 | E4 | 40 | mg/kg | BIDx14 | po | 28 | Vehicle | 6 |
| 6 | E9 | 25 | mg/kg | BIDx14 | po | 28 | Vehicle | 6 |
| 7 | E9 | 50 | mg/kg | BIDx14 | po | 28 | Vehicle | 6 |
| 8 | E10 | 30 | mg/kg | BIDx14 | po | 28 | Vehicle | 6 |
| 9 | Rat IgG2a isotyp | 10 | mg/kg | 3q7d | i.p. | 12 | Vehicle | 10 |
| | control (clone 2A3) | | | | | | | |
| 10 | Anti-mPD-1 | 10 | mg/kg | 3q7d | i.p. | 12 | Vehicle | 10 |
| | (clone RMP1-14) | | | | | | | |
| 11 | Vehicle | 10 | mg/kg | BIDx28 | po | 56 | | 10 |
| 12 | C7 | 100 | mg/kg | BIDx28 | po | 56 | Vehicle | 10 |
| 13 | E4 | 40 | mg/kg | BIDx28 | po | 56 | Vehicle | 10 |
| 14 | E9 | 25 | mg/kg | BIDx28 | po | 56 | Vehicle | 10 |
| 15 | E9 | 50 | mg/kg | BIDx28 | po | 56 | Vehicle | 10 |
| 16 | E10 | 30 | mg/kg | BIDx28 | po | 56 | Vehicle | 10 |

*= 10% DMSO + 5% Solutol + 40% PEG + 45% water

Additionally, tumour and blood samples were used to analyse various immune-response markers, for example those set forth in Table 5.2 below (and as described below in connection with Table 8D in Example 8 of WO2020/083926)).

TABLE 5.2
Example immune-phenotype markers.

| | |
|---|---|
| T cell panel | Myeloid cell panel |

| Target | Clone | Target | Clone |
|---|---|---|---|
| CD45 | 30-F11 | CD45 | 30-F11 |
| CD3e | 145-2C11 | CD3e | 145-2C11 |
| CD4 | RM4-5 | CD11b | M1/70 |
| CD8 | 53-6.7 | F4/80 | BM8 |
| CD25 | PC61 | Ly6C | AL-21 |
| FoxP3 | FJK-16s | Ly6G | 1A8 |
| IFNy | XMG1.2 | CD206 | C068C2 |
| GrzB | NGZB | MHC-II | 10-3.6 |
| CD69 | H1.2F3 | CD49b | DX5 |
| CD107a | 1D4B | CD335 | 29A1.4 |
| Live/dead | (Zombie Dye) | Live/dead | (Zombie Dye) |

The applicant described that it was surprised to observe that twice daily application of C7 and E9 showed 60 to 80% TGI (Table 5.3). The applicant also described also observed the very surprising result that the anti-cancer efficacy of those compounds was even superior to anti-PD-1 therapy, which only resulted in 4% TGI (FIGS. 4A and 4B, and Table 5.3). Indeed, a tumour growth inhibition effect with the inhibitors was seen in almost all of the treated mice of the compound treated cohorts (FIGS. 4C to 4E), in comparison to only few responding animals in the anti-PD1 treated and control groups (FIGS. 4F to 4H).

Indeed, E9, dosed at only 25 mg/kg BID, consistency showed tumour growth inhibition across all individual mice in the treatment group (average TGI=72%), compared to increased variability in a comparative study with dasatinib (A8, dosed at 30 mg/kg QD), where in some cases individual mice in the dasatinib treatment (average TGI=56%) grew significant tumours (FIG. 5).

Furthermore, and providing further evidence that kinase inhibitor E9 exhibited drug-like properties when administered orally, no direct treatment related side effects and clinical signs, such as loss of body weight were observed.

TABLE 5.3
In-vivo data of kinase inhibitors E9 (and
other inhibitors) compared to PD-1.

| | Tumour | Body | Non- |
|---|---|---|---|
| | growth | weight | treatment |
| | inhibition | increase | related |
| Treatment | (%) | (%) | death |
| Rat IgG2a isotyp | 0 | 25.4 | 2/16 |
| control (10 mg/Kg) | | | |
| Anti-mPD-1, (10 mg/Kg) | 4 | 30.6 | 1/16 |
| Vehicle (10 mg/Kg) | 0 | 24.0 | 2/16 |
| C7 (100 mg/Kg, BID) | 78 | 1.0 | 3/16 |
| E4 (40 mg/Kg, BID) | 64 | 12.0 | 3/16 |
| E9 (25 mg/Kg, BID) | 74 | 16.8 | 2/16 |
| E9 (50 mg/Kg, BID) | 69 | 18.7 | 3/16 |
| E10 (30 mg/Kg, BID) | 46 | 19.8 | 0/16 |

In addition, these kinase inhibitors (in particular, compound E9) demonstrated a surprisingly significant effect on the immune cells present in the tumour microenvironment (data not shown, as described in Example 8.1 of PCT/EP2021/060338, and corresponding FIG. 21 thereof).

### Example 6 [Comparative]: Research-Scale/Grade Synthesis of Compound E9 and of the Other Prior Art Compounds A8, B3, C7 and D9

Compounds A8, B3, C7, and D9 were synthesised as described in WO2020/083926), and Compound E9 was synthesised as is described in PCT/EP2021/060338 (unpublished on the priority date of the present invention).

The synthesis of Compound E9 described in PCT/EP2021/060338 is one suitable for bench/research-scale/grade, and the scheme and procedure used thereon is summarised in FIG. 2. Such a scheme and procedure has certain limitations, including in terms of overall % yield of compound E9, time, and/or scalability/absolute yield of compound E9, as well as overall cost per unity amount of compound E9.

For example, ester hydrolysis occurs as a second step, and the specific and novel intermediate 46 is introduced as a third step. In this prior-art scheme, addition of the commercially (and cheaply obtainable) precursor methylpyrazine (49) occurs as the final step.

Indeed, intermediate 46 (important for the synthesis of compound E9), was synthesised by a method with a yield of only 72%, and was produced as a yellow-discoloured solid.

Briefly, (and as described in PCT/EP2021/060338):

### Compound E9:

### Synthesis of N-(2-chloro-4-(fluoromethyl)thiophen-3-yl)-2-((2-methyl-6-(4-methylpiperazin-1-yl)pyrimidin-4-yl)amino)thiazole-5-carboxamide (E9)

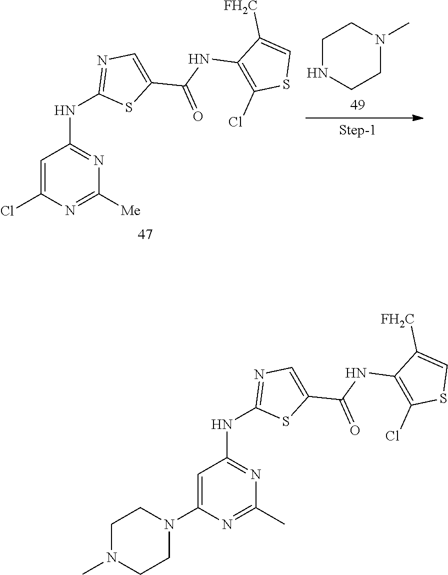

1-methylpiperazine 49 (263 mg, 2.63 mmol, 5.0 eq.) and DIPEA (27 mg, 0.21 mmol, 0.4 eq.) were added to a well stirred solution of 2-((6-chloro-2-methylpyrimidin-4-yl)amino)-N-(2-chloro-4-(fluoromethyl)thiophen-3-yl) thiazole-5-carboxamide 47 (220 mg, 0.52 mmol, 1.0 eq.) in n-BuOH. The reaction temperature was then raised to 100° C. for 3 h. The reaction mixture was concentrated under vacuum and the obtained residue was purified by RP C18 to yield N-(2-chloro-4-(fluoromethyl)thiophen-3-yl)-2-((2-methyl-6-(4-methylpiperazin-1-yl)pyrimidin-4-yl)amino) thiazole-5-carboxamide (E9, FIG. 1E) (55 mg) as a white solid.

Analytical data for compound E9 as synthesised by the scheme described in PCT/EP2021/060338 (unpublished on the priority date of the present invention). is shown in Table 6.1.

TABLE 6.1
Synthesis of compound E9 by the scheme
described in PCT/EP2021/060338.

| | Mol. Wt. | |
|---|---|---|
| Compound | LCMS: m/z | |
| Number | [M + H]⁺ | ¹H NMR |
| E9 | 482.18 | ¹H NMR (300 MHz, DMSO-d6): δ 11.49 (s, |
| | 99.57% | 1H), 9.94 (s, 1H), 8.21 (s, 1H), 7.67 (d, J = |
| | (4.25 min) | 3.3 Hz, 1H), 6.05 (s, 1H), 5.25(d, J = 47.7 |
| | | Hz, 2H), 3.51 (bs, 4H), 2.41 (s, 3H), 2.37 |
| | | (bs, 4H), 2.21 (s, 3H). |

### Compound 47:

### 2-((6-chloro-2-methylpyrimidin-4-yl)amino)-N-(2-chloro-4-(fluoromethyl)thiophen-3-yl) thiazole-5-carboxamide (47)

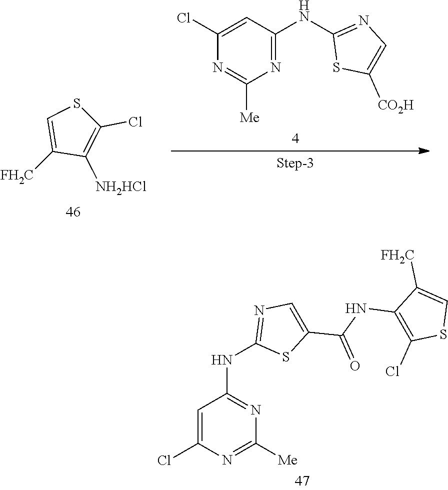

To a suspension of 2-chloro-4-(fluoromethyl)thiophen-3-amine hydrochloride 46 (100 mg, 0.49 mmol, 1.0 eq.) and 2-((6-chloro-2-methylpyrimidin-4-yl)amino)thiazole-5-carboxylic acid 4 (173 mg, 0.64 mmol, 1.3 equiv) in acetonitrile (3 mL) was added DIPEA (224 mg 1.73 mmol, 3.5 eq.) and TCFH (160 mg, 0.57 mmol, 1.15 eq.) at room temperature under a nitrogen atmosphere. The reaction mixture was stirred at room temperature for 24 hr. Reaction was monitored by LCMS. The reaction mixture was concentrated under vacuum and obtained residue was passed through column to afford crude 2-((6-chloro-2-methylpyrimidin-4-yl)amino)-N-(2-chloro-4-(fluoromethyl) thiophen-3-yl) thiazole-5-carboxamide 47 (220 mg, purity ca. 72%) as a yellow solid.

LCMS: m/z 418.07 [M+H]⁺, (3.33 min).

### Compound 46:

### Step-1: tert-butyl (2-chloro-4-(fluoromethyl)thiophen-3-yl)carbamate (45)

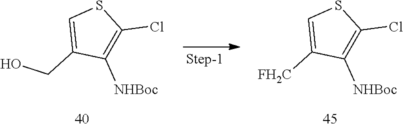

Diethylaminosulfur trifluoride (DAST) (0.9 ml, 6.82 mmol, 2.0 eq.) was added drop-wise to the solution of tert-butyl (2-chloro-4-(hydroxymethyl)thiophen-3-yl)carbamate 40 (0.9 g, 3.41 mmol, 1.0 eq.) in dichloromethane (20 mL) at 0° C. The reaction mixture was stirred for 3 h at ambient temperature and then cooled to 0° C., before adding a saturated aqueous sodium bicarbonate solution until the neutral state was reached. The mixture was then extracted with dichloromethane. The organic phase was distilled under low pressure and the obtained residue was purified by column chromatography that offered the tert-butyl (2-chloro-4-(fluoromethyl)thiophen-3-yl)carbamate 45 (408 mg, 45% yield) as a colourless liquid.

¹H NMR (300 MHz, CDCl₃): δ 7.14 (d, J=2.7 Hz, 1H), 6.09 (bs, 1H), 5.27 (d, J=47.7 Hz, 2H), 1.49 (s, 9H). LCMS: m/z 264.19 [M+H]⁺, 98.14% (3.75 min).

### Step-2: 2-chloro-4-(fluoromethyl)thiophen-3-amine hydrochloride (46)

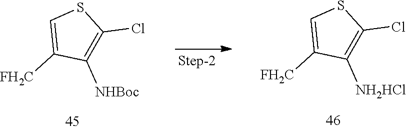

To a solution of tert-butyl (2-chloro-4-(fluoromethyl)thiophen-3-yl)carbamate 45 (266 mg, 1.0 mmol, 1.0 eq.) in anhydrous dioxane (1.0 mL) was added 4.0 N HCl in dioxane (2.5 mL, 10.0 mmol, 10.0 eq.) at room temperature under a nitrogen atmosphere. The reaction mixture was stirred at room temperature for 6 h. Solid precipitated was filtered and washed with hexane to afford 2-chloro-4-(fluoromethyl)thiophen-3-amine hydrochloride 46 (157 mg, 78%) as an off-white solid.

### Compound 40:

### Step-1: methyl 3-((2-methoxy-2-oxoethyl)thio)propanoate

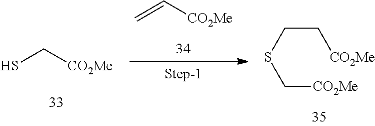

Methyl acrylate 34 (99.16 mL, 1.1 mol) was slowly added to a solution of methyl 2-mercaptoacetate 33 (91 mL, 1 mol) and piperidine (2 mL) and stirred the reaction mixture at temperature 50° C. for 2 h. After complete disappearance of starting material by TLC, excess methyl acrylate and piperidine were distilled under high vacuum to give methyl 3-((2-methoxy-2-oxoethyl)thio)propanoate 35 (190 g, 99% yield) as a colourless viscous liquid oil.

¹H NMR (300 MHz, CDCl₃): δ 3.73 (s, 3H), 3.69 (s, 3H), 3.25 (s, 2H), 2.9 (t, J=7.2 Hz, 2H), 2.64 (t, J=7.2 Hz, 2H).

### Step-2: methyl 4-oxotetrahydrothiophene-3-carboxylate

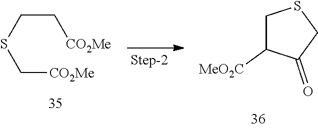 [SMILES: COC(=O)CCSCC(=O)OC ; COC(=O)C1CSCC1=O]

To a solution of 60% NaH (13.2 g, 330 mmol, 1.1 eq.) in THF, methyl 3-((2-methoxy-2-oxoethyl) thio)propanoate 35 (58 g, 300 mmol, 1.0 eq.) in dry THF (800 mL) was added slowly within 2 h, and the reaction mixture was refluxed for 5 h. Then, the solvent was concentrated under reduced pressure, and H₂O (300 mL) was added to the residue. The pH value of the solution was adjusted to 1 by cold 1M HCl solution and extracted with DCM. The combined organic layers were dried over Na₂SO₄ and concentrated under vacuum. The residue was purified by column chromatography to yield methyl 4-oxotetrahydrothiophene-3-carboxylate 36 (17 g, 35%) as colourless viscous oil.

¹H NMR (300 MHz, CDCl₃): δ 10.94 (s, 1H), 3.75-3.82 (m, 7H).

### Step-3: methyl 4-aminothiophene-3-carboxylate

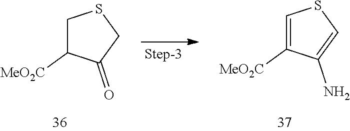 [SMILES: COC(=O)C1CSCC1=O ; COC(=O)c1cscc1N]

The mixture of methyl 4-oxotetrahydrothiophene-3-carboxylate 36 (3.9 g, 24.4 mmol, 1.0 eq.), hydroxylamine hydrochloride (1.69 g, 24.4 mmol, 1.0 eq.) and acetonitrile (20 mL) was stirred under reflux temperature for 1 h. The reaction mixture was then cooled and the solid which separated was filtered off and washed with dry ether to afford the methyl 4-aminothiophene-3-carboxylate 37 (2.9 g, 75%) as colourless viscous liquid oil.

### Step-4: methyl 4-((tert-butoxycarbonyl)amino)thiophene-3-carboxylate

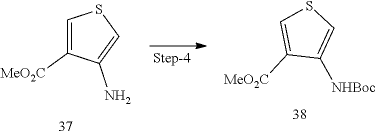 [SMILES: COC(=O)c1cscc1N ; COC(=O)c1cscc1NC(=O)OC(C)(C)C]

To a solution of methyl 4-aminothiophene-3-carboxylate 37 (6.57 g, 41.8 mmol, 1.0 eq.) in 1,4-dioxane (25 mL) at 0° C. was added 5% aqueous Na₂CO₃ solution, followed by a solution of di-tert-butyl dicarbonate (18.26 g, 83.7 mmol, 2.0 eq.) in dioxane (25 mL). The cold bath was removed and the reaction mixture was allowed to room temperature and stirred for 24 hours, then diluted with water and EtOAc. The aqueous layer was extracted with EtOAc and the combined organic layers were washed with water and brine, dried over Na₂SO₄, filtered and concentrated in vacuum. The residue obtained was purified using column chromatography to provide methyl 4-((tert-butoxycarbonyl)amino)thiophene-3-carboxylate 38 (6.45 g, 60% yield).

¹H NMR (400 MHz, DMSO-d6): δ 9.06 (s, 1H), 8.34 (d, J=3.6 Hz, 1H), 7.58 (s, 1H), 3.83 (s, 3H), 1.47 (s, 9H).

### Step-5: methyl 4-((tert-butoxycarbonyl)amino)-5-chlorothiophene-3-carboxylate

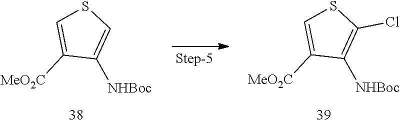 [SMILES: COC(=O)c1cscc1NC(=O)OC(C)(C)C ; COC(=O)c1csc(Cl)c1NC(=O)OC(C)(C)C]

To a solution of methyl 4-((tert-butoxycarbonyl)amino)thiophene-3-carboxylate 38 (16 g 62.2 mmol, 1.0 eq.) in glacial acetic acid (80 mL) was added N-chlorosuccinimide (8.3 g 62.2 mmol, 1.0 eq.) and the resultant reaction mixture was stirred at 50° C. for 45 minutes. Acetic acid was distilled under reduced pressure and the residue obtained was treated with water. The mixture was made alkaline with sodium hydroxide solution and then extracted with ethyl acetate. Organic extracts were combined, washed with water, dried over Na₂SO₄, concentrated and purified by column chromatography to offered methyl 4-((tert-butoxycarbonyl)amino)-5-chlorothiophene-3-carboxylate 39 (14.4 g, 80% yield) as colourless liquid.

¹H NMR (300 MHz, DMSO-d6): δ 8.78 (s, 1H), 8.13 (s, 1H), 3.73 (s, 3H), 1.41 (s, 9H). LCMS: m/z 192.16 [M−100]⁺, 99.40% (3.71 min).

### Step-6: tert-butyl (2-chloro-4-(hydroxymethyl)thiophen-3-yl)carbamate

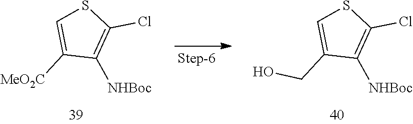 [SMILES: COC(=O)c1csc(Cl)c1NC(=O)OC(C)(C)C ; CC(C)(C)OC(=O)Nc1c(CO)csc1Cl]

1M DIBAL in toluene (51.4 ml, 51.4 mmol, 3.0 eq.) was added slowly to the solution of methyl 4-((tert-butoxycarbonyl) amino)-5-chlorothiophene-3-carboxylate 39 (5 g, 17.14 mmol, 1.0 eq.) in DCM at 0° C. and the reaction mixture was stirred at room temperature for 4 h. The reaction mixture was diluted with 2N NaOH solution and extracted with DCM. The combined organic layers were concentrated and purified by column chromatography to give tert-butyl (2-chloro-4-(hydroxymethyl)thiophen-3-yl)carbamate 40 (4 g, 88% yield).

¹H NMR (300 MHz, CDCl₃): δ 7.12 (s, 1H), 6.18 (s, 1H), 4.43 (s, 2H), 1.51 (s, 9H). LCMS: m/z 190.13 [M−56−18]⁺, (3.14 min).

### Compound 4:

### Step-1: ethyl 2-((6-chloro-2-methylpyrimidin-4-yl)amino)thiazole-5-carboxylate (3)

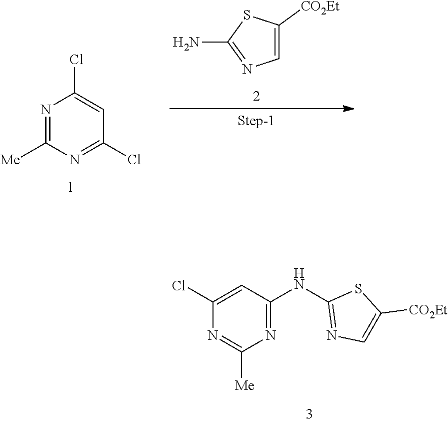 [SMILES: Cc1nc(Cl)cc(Cl)n1 ; CCOC(=O)c1cnc(N)s1 ; CCOC(=O)c1cnc(Nc2cc(Cl)nc(C)n2)s1]

To a solution of 4,6-dichloro-2-methylpyrimidine 1 (10 g, 61.4 mmol, 1.0 eq.) and ethyl 2-aminothiazole-5-carboxylate 2 (10.6 g, 61.4 mmol, 1.0 eq.) in DMF (210 ml) at 0° C. under inert atmosphere was added 60% sodium hydride (4.9 g, 122.8 mmol, 2.0 eq.) in portions and the reaction mixture was slowly warmed to room temperature and stirred for 3 days. Excess of NaH was quenched by the addition of saturated ammonium chloride solution and the reaction mixture was diluted with water (3000 ml) and stirred for 1 h at room temperature. Obtained precipitate was filtered off and air dried to get ethyl 2-((6-chloro-2-methylpyrimidin-4-yl)amino)thiazole-5-carboxylate 3 (18 g, 98% yield) as a beige solid.

¹H NMR (400 MHz, CDCl₃) δ 1.34-1.51 (t, 3H), 2.75 (s, 3H), 4.41 (q, J=7.1 Hz, 2H), 6.73 (s, 1H), 8.14 (s, 1H). LCMS: m/z=299.17 [M+H]⁺, (3.45 min).

### Step-2: 2-((6-chloro-2-methylpyrimidin-4-yl)amino)thiazole-5-carboxylic acid (4)

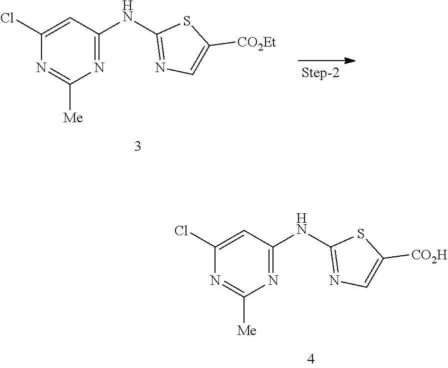 [SMILES: CCOC(=O)c1cnc(Nc2cc(Cl)nc(C)n2)s1 ; Cc1nc(Cl)cc(Nc2ncc(C(=O)O)s2)n1]

To a suspension of ethyl 2-((6-chloro-2-methylpyrimidin-4-yl)amino)thiazole-5-carboxylate 3 (36.0 g, 120.5 mmol, 1.0 eq.) in methanol (350 ml) and water (150 ml) was added sodium hydroxide (38.6 g, 964 mmol, 8.0 eq.) at room temperature and the mixture was stirred for 16 hrs. LCMS analysis showed complete conversion of starting material to product. The reaction mixture was concentrated to remove most of the solvent and then the aqueous layer was acidified using 6M aqueous HCl. The obtained precipitate was filtered off, washed with water and dried under high vacuum for 3 days to afford 2-((6-chloro-2-methylpyrimidin-4-yl)amino)thiazole-5-carboxylic acid 4 (27.0 g, 83% yield) as a beige coloured powder.

¹H NMR (400 MHz, DMSO-d₆) δ 2.57 (s, 3H), 6.93 (s, 1H), 8.04 (s, 1H), 12.46 (bs. 1H). LCMS: m/z=271.05 [M+H]⁺, 99.19% (2.51 min).

### General Methods and Materials Used for Compound Syntheses According to Example 6:

MPLC purification was performed using a Biotage Isolera Four system, using KP-Sil cartridges with technical grade organic solvents, i.e. dichloromethane and methanol, 3-4 N NH₃ in MeOH. A gradient of DCM to 3 N NH₃ (in MeOH) from 0% to 25% over 10 CV was used for the purification of the final compounds.

¹H NMR spectra were recorded on Bruker DPX 400 MHz spectrometers and are reported in ppm with the solvent resonance employed as the internal standard [CDCl₃ at 7.26 ppm, DMSO-d6 at 2.50 ppm]. Peaks are reported as (s=singlet, d=doublet, t=triplet, q=quartet, m=multiplet or unresolved, bs=broad signal, coupling constant(s) in Hz, integration).

Reverse phase HPLC was performed on a Shimadzu HPLC system using following system [solvent A: acetonitrile, solvent B: 0.1% formic acid in water]. Formic acid was used as HPLC grade. All the separations were performed at ambient temperatures. For analytical RP-HPLC analysis [Interchim: Uptisphere Strategy 100 Å, 5 μm, 100×4.6 mm], the flow rate was 1.0 ml·min⁻¹; injection volume: 20 μL, detection wavelengths: 220 nm and 254 nm. The following gradient was used: 2.0 min 100% B, over 8 min to 10% B, 5 min 10% B.

LC-MS spectra were recorded on a Dionex Ultimate 3000 system using the following system [solvent A: acetonitrile, solvent B: 0.1% formic in water]. Formic acid was used as HPLC grade. All the separations were performed at ambient temperatures. For analytical RP-HPLC analysis [Interchim: Uptisphere Strategy C18, 2.6 μm, 50×4.6 mm], the flow rate was 1.0 ml·min⁻¹; detection wavelengths: 220 nm and 254 nm. The following gradient was used: 90% B, over 5 min to 5% B. The MS was recorded with the following settings: Dionex Surveyor MSQ plus, ESI+, Probe T(° C.) 350, Cone 30 (v), Needle (KV) 3.0.

Abbreviations: Boc₂O: di-tert-butyl decarbonate; 1-BuOH: 1-butanol; cHex: cyclohexane; CV: column value; d: doublet (NMR); d: days; dd: doublet of doublets; DCM=CH₂Cl₂: dichloromethane; DIPEA: N,N-diethylisopropyl amine; DMF: N,N-dimethylformamide; DMSO: dimethyl sulphoxide; EDC HCl: 1-Ethyl-3-(3-dimethylaminopropyl)carbodiimide; equiv.: equivalents; Et₂O: diethyl ether; EtOAc: ethyl acetate; g: gram; h: hours; H: proton; HCl: hydrochloric acid; H₂O: water; HOBT: 1-hydroxybenzotriazol; Hz: Hertz; IPA: iso-propanol; J: scalar ¹H-¹H coupling constant; K₂CO₃: potassium carbonate; KOH: potassium hydroxide; LC-MS: liquid chromatography-mass spectrometry; m: multiplet; M: molar; mAU: milliabsorption units; Me: methyl; MeCN: acetonitrile; MeOH: methanol; mg: milligram; MHz: mega Hertz; min: minutes; pw: microwave; N₂: nitrogen; NaH: sodium hydride; NaHCO₃: sodium bicarbonate; NaOH: sodium hydroxide; Na₂SO₄: sodium sulphate; NBS: A-bromo succinimide; NCS: N-chloro succinimide; NMR: nuclear magnetic resonance; PdCl₂(dppf): [1,1′-Bis(diphenylphosphino)ferrocene]dichloropalladium (II); Pd₂dba₃: Tris(dibenzylideneacetone)dipalladium(0); quant.: quantitative; Rf: retention factor (TLC); rt: room temperature; s: singlet; SiO₂: silica; TCFH: tetramethylchloroformamidinium hexafluorophosphate; TFA: trifluoro acetic acid; THF: tetrahydrofuran; TLC: thin layer chromatography; XantPhos: 4,5-Bis(diphenylphosphino)-9,9-dimethylxanthene.

### Example 7: New Scheme and Process for the Preparation of Compound E9

As described below, the applicant has developed an improved 4-step scheme for the synthesis of compound E9, such scheme, and the processes used to conduct such scheme show advantages over the scheme for synthesising compound E9 as described in PCT/EP2021/060338 (and as summarised in the comparative Example 6 above), for example in terms of the scale of synthesis and molar-efficiency of incorporation of the key intermediate compound 46.

### Compound 3:

### Step 1: Synthesis of ethyl 2-((6-chloro-2-methylpynmidin-4-yl)amino)thiazole-5-carboxylate (3)

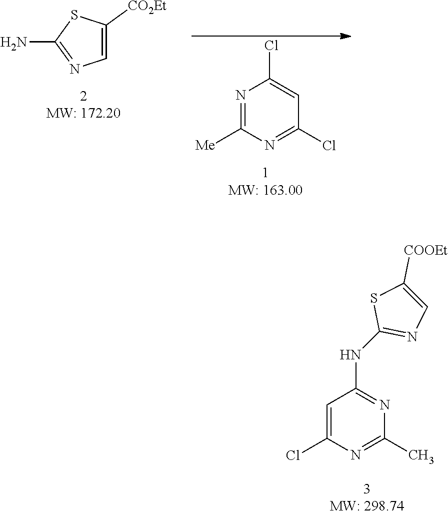 [SMILES: CCOC(=O)c1cnc(N)s1 ; Cc1nc(Cl)cc(Cl)n1 ; CCOC(=O)c1cnc(Nc2cc(Cl)nc(C)n2)s1]

In a 10 L jacketed reactor, 1 (432 g, 1.1 eq.) was dissolved in DMF (2.1 L) at 20° C. 2 (415 g, 1 eq.) was charged, the resulting mixture was cooled to 5° C. and cesium carbonate (1.6 kg, 2 eq.) was charged in 10 portions. The mixture was warmed to 20° C. and it was stirred at 20° C. for 22 hours. The mixture was cooled to 10° C. and water (6.2 L) was added. The mixture was warmed to 20° C. and the slurry was stirred at 20° C. for 15 hours. The solid was recovered by filtration and it was dried under vacuum at 45° C. to give 530 g (73.6% mol yield) of 3 as yellow powder.

Compound 3: Purity (HPLC, 30 minutes method, 325 nm): 98.38% a/a. 1H NMR (DMSO-d6, 400 MHz) ppm: 1.30 (t, 3H), 2.59 (s, 3H), 4.29 (q, 2H), 6.94 (s, 1H), 8.13 (s, 1H), 12.36 (br s, 1H). UPLC-MS [M+H]+: 299.01 m/z. Residue on ignition: 0.2% w/w. Thermogravimetric analysis: 0.4% w/w.

### Ester Intermediate Having the Formula Ia:

### Step 2: Synthesis of ethyl 2-((6-(4-methylpiperazin-1-yl)-2-methylpyrimidin-4-yl)amino)thiazole-5-carboxylate (Ia)

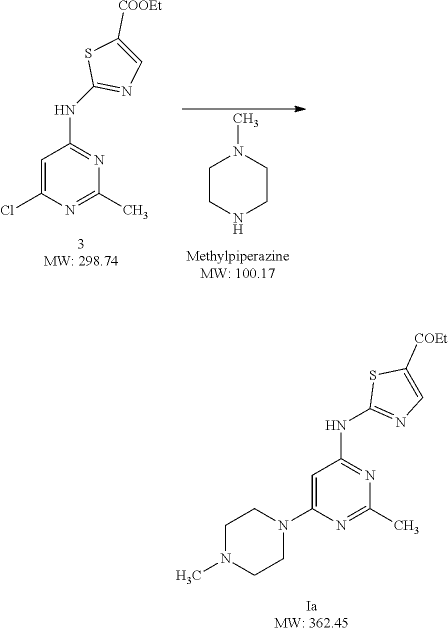

In a 20 L jacketed reactor, 3 (1.21 kg, 1 eq.) was suspended in n-butanol (12.1 L) at 20° C. Me-piperazine (673 ml, 1.5 eq.) and DIPEA (1.4 L, 2 eq.) were sequentially added and the mixture was heated at reflux. The mixture was stirred at reflux for 5 hours and then it was cooled down to 20° C. Water (2.4 L) was charged and the slurry was aged at 20° C. for three hours. The solid was recovered by filtration and it was dried under vacuum at 50° C. for 30 hours to give 1.29 kg (88% mol yield) of Ia as white powder.

Ester intermediate Ia: Purity (HPLC, 30 minutes method, 325 nm): 97.84% a/a. 1H NMR (Acetic acid-d4, 600 MHz) ppm: 1.37 (t, 3H), 2.53 (s, 3H), 2.94 (s, 3H), 3.05 (br s, 2H), 3.47 (br s, 2H), 3.77 (br s, 2H), 4.36 (q, 2H), 4.55 (br s, 2H), 6.15 (s, 1H), 8.02 (s, 1H). UPLC-MS [M+H]+: 363.15 m/z. Residue on ignition: 0.0% w/w. Thermogravimetric analysis: 0.2% w/w.

In an alternative procedure, the following protocol was used: in a 0.5 L jacketed reactor, 3 (20 g, 1 eq.) was suspended in n-butanol (180 ml) at 20° C. Me-piperazine (11.14 ml, 1.5 eq.) and DIPEA (23.3 ml, 2 eq.) were sequentially added and the mixture was heated at reflux. The mixture was stirred at reflux for 4 hours and then it was cooled down to 20° C. Water (40 ml) was charged and the slurry was aged at 20° C. for two hours. The mixture was heated to 60° C., stirred at 60° C. and then cooled to 20° C. The cold-hot cycle was repeated for two more times. The solid was recovered by filtration and it was dried under vacuum at 50° C. for 30 hours to give 20.5 g (85% mol yield) of Ia as white powder.

A comparative example 1 as shown in Table 7.1 was performed which demonstrates that hot and cold cycles increase product yield. Ia was suspended in 1-BuOH/water 8.33/1.6 solution. The mixtures were aged overnight at 20° C. or they were treated with cold-hot cycles (20° C.-60° C.-20° C.). The improvement in the filtration rate were measured by observing the overall filtration time.

TABLE 7.1
Comparative Example 1

| | | | Filtration time overall (mins) | | | | |
|---|---|---|---|---|---|---|---|
| | | Slurry | PTFE filter (porosity 20 μm) | | | | |
| INT-14 | | conditions | 5 mL at | 7 mL at | >7 mL at 500 mbar | Output | Uncorrected |
| input (g) | Solvent | (° C.) | 800 mbar | 600 mbar | (includes washes) | (g) | yield % |
| 1.00 | 1-ButOH/water | 20 | 00:12:04 | 00:17:53 | 00:40:55 | 0.982 | 98.2 |
| | 8.33:1.6 | ageing o/n | | | | | |
| 1.00 | 1-ButOH/water | 20-60-20 | 00:07:40 | 00:10:33 | 00:15:18 | 0.995 | 99.5 |
| | 8.33:1.6 | 3 cycles o/n | | | | | |

### Carboxylic Acid Intermediate Having the Formula I:

### Step 3: Synthesis of 2-((6-(4-methylpiperazin-1-yl)-2-methylpyrimidin-4-yl)amino)thiazole-5-carboxylic acid (I)

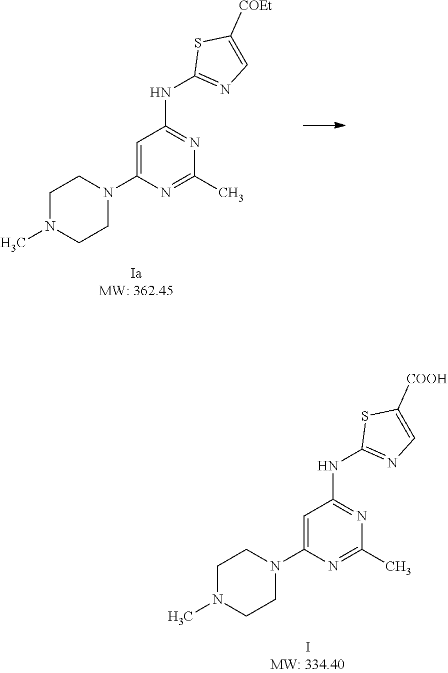

In a 20 L jacketed reactor, solid NaOH (566 gr, 4 eq.) was dissolved in water (10.3 L) at 20° C. Ia (1.28 kg, 1 eq.) was charged and the mixture was heated to 60° C. The mixture was stirred at 60° C. for 32 hours and then it was cooled down to 20° C. Aq. HCl 6 M (2.3 L) was charged and the mixture was stirred at 20° C. for 15 hours. Aq. HCl 6 M (150 ml) was charged and the mixture was stirred at 20° C. for 1 hour. The solid was isolated by filtration and it was dried under vacuum at 50° C. for 20 hours to give 1.1 kg (93% mol yield) of I as white powder.

Carboxylic acid intermediate I: Purity (HPLC, 30 minutes method, 325 nm): 99.29% a/a. 1H NMR (Acetic acid-d4, 400 MHz) ppm: 2.53 (s, 3H), 2.95 (s, 3H), 3.06 (br s, 2H), 3.48 (br s, 2H), 3.78 (br s, 2H), 4.55 (br s, 2H), 6.12 (s, 1H), 8.06 (s, 1H). UPLC-MS [M+H]+: 335.07 m/z. Residue on ignition: 1.0% w/w. Thermogravimetric analysis: 1.1% w/w.

In an alternative procedure, the following protocol was used: in a 0.5 L jacketed reactor, solid NaOH (8.83 gr, 4 eq.) was dissolved in water (160 ml) at 20° C. Ia (20 g, 1 eq.) was charged and the mixture was heated to 60° C. The mixture was stirred at 60° C. for 22 hours and then it was cooled down to 20° C. Aq. HCl 6 M (36 ml) was charged and the mixture was stirred at 20° C. for 8 hours. Acetonitrile (48 ml) was charged and the mixture was heated to 60° C. The mixture was stirred at 60° C. and then it was cooled to 20° C. The mixture was stirred at 20° C. overnight. The solid was isolated by filtration and it was dried under vacuum at 50° C. for 20 hours to give 15 g (81% mol yield) of I as white powder.

A comparative experiment 2 as shown in Table 7.2 was conducted: compound I was suspended in acetonitrile/water solutions. The mixtures were aged overnight at 20° C. or they were treated with cold-hot cycles (20° C.-60° C.-20° C.). The improvement in the filtration rate were measured by observing the overall filtration time.

TABLE 7.2
Comparative Example 2

| | | | Filtration time overall (mins) | | | | |
|---|---|---|---|---|---|---|---|
| | | Slurry | PTFE filter (porosity 20 μm) | | | | |
| INT-15 | | conditions | 5 mL at | 10 mL at | >10 mL at 500 mbar | Output | Uncorrected |
| input (g) | Solvent | (° C.) | 800 mbar | 600 mbar | (includes washes) | (g) | yield % |
| 1.00 | water | 20-60-20 | 00:11:20 | 00:20:40 | 00:29:45 | 0.987 | 98 |
| | | 3 cycles o/n | | (includes one wash) | | | |
| 1.00 | water/CH3CN 80:20 | 20-60-20 | 00:02:45 | 00:05:08 | 00:11:55 | 1.030 | 100 |
| | | 3 cycles o/n | | | | | |
| 1.00 | water/CH3CN 70:30 | 20-60-20 | 00:01:00 | 00:01:54 | 00:05:11 | 1.000 | 100 |
| | | 3 cycles o/n | | | | | |
| 1.00 | water/CH3CN 70:30 | 20-60-20 | 00:00:40 | 00:01:36 | 00:04:58 | 0.970 | 97 |
| | | 1 cycle + | | | | | |
| | | ageing o/n | | | | | |
| 1.00 | water/CH3CN 70:30 | 20 | 00:00:43 | 00:01:51 | 00:05:25 | 0.999 | 99 |
| | | ageing o/n | | | | | |

### Compound E9:

### Step 4: Synthesis of N-(2-chloro-4-(fluoromethyl)thiophen-3-yl) 2-((2-methyl-6-(4-methylpiperazin-1-yl)pyrimidin-4-ylamino)thiazole-5-carboxamide (E9)

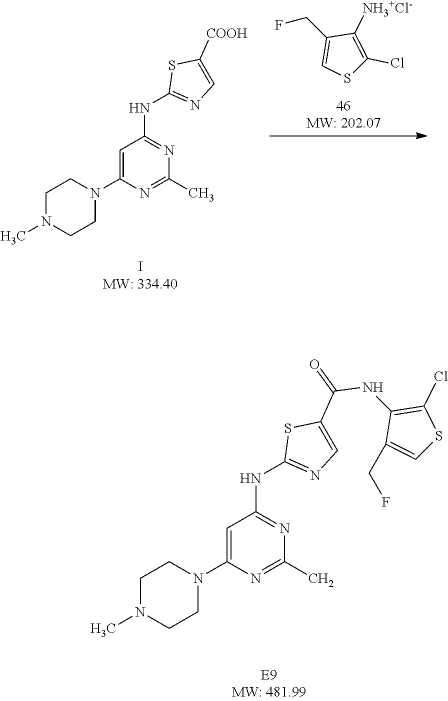

In a 20 L jacketed reactor, I (550 g, 1 eq.) was suspended in acetonitrile (16.5 L). The mixture was heated to reflux and concentrated to 8.25 L. The mixture was cooled down to 20° C. and TCFH (660 g, 1.43 eq.) and 46 (382 g, 1.15 eq.) were sequentially charged. The mixture was heated to 40° C. and DIPEA (745, ml, 2.6 eq.) was charged in 1.5 hour. The mixture was stirred 10 minutes at 40° C. and it was cooled down to 20° C. Aq. NaOH 0.25 M (11 L) was charged in 30 minutes and the mixture was stirred at 20° C. for 22 hours. The solid was recovered by filtration and it was dried under vacuum at 50° C. for 18 hours to give 600 g (75.7% mol yield) of E9 as yellow powder.

Compound E9: Purity (HPLC, 30 minutes method, 325 nm): 96.63% a/a. 1H NMR (DMSO-d6, 400 MHz) ppm: 2.21 (s, 3H), 2.35-2.39 (m, 4H), 2.41 (s, 3H), 3.47-3.55 (m, 4H), 5.25 (d, 2H), 6.05 (s, 1H), 7.68 (d, 1H), 8.22 (s, 1H), 9.96 (s, 1H), 11.51 (s, 1H). UPLC-MS [M+H]+: 482.14 m/z. Residue on ignition: 0.1% w/w. Thermogravimetric analysis: 1.0% w/w.

### Example 8: New Reaction Conditions for the Preparation of Compound 46 from Compound 45

As described below, the applicant has developed an improved reaction for synthesising compound 46 from compound 45. Such process shows advantages over the reactions for synthesising compound 46 from compound 45 as is described in PCT/EP2021/060338 (and as summarised in the comparative Example 6 above), for example in terms of the final scale of synthesis, yield and purity of the synthesis of compound 46, a characteristic thiophene-based key amino intermediate in the preparation of the SIK3 inhibitor compound E9.

In particular, the new reaction conditions using ethyl acetate (EA) of the present applicant, achieves 90% mol yield compared to only 78% yield with of the reaction conditions shown in comparative Example that uses dioxane.

### Amino Intermediate 46:

### 2-chloro-4-(fluoromethyl)thiophen-3-amine hydrochloride

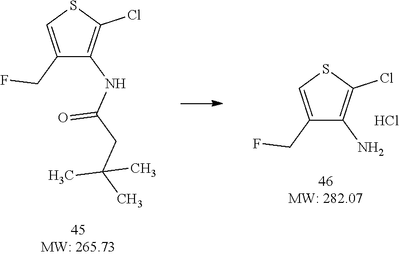 [SMILES: CC(C)(C)CC(=O)Nc1c(CF)csc1Cl ; Nc1c(CF)csc1Cl ; Cl]

In a round bottom flask, 45 (10 g, 1.1 eq.) was added to ethyl acetate (EA) (30 ml) at 25° C. HCl 4M in EA (4 eq.) was charged and the mixture was stirred at 25° C. for at least 4 hours. The resulting suspension was filtered to isolate the desired product and the wet cake was dried under vacuum at 30° C. for 18 hours to give 7 g (90% mol yield) of amino intermediate 46 as off-white solid.

Amino intermediate E9: Purity: 98.5%. 1H NMR (DMSO-d6, 500 MHz) ppm: 5.47 (d, 2H), 7.66 (d, 1H). UPLC-MS [M+H]+: 166.0 m/z.

Compound 45 may be prepared as described in PCT/EP2021/060338 (and as summarised in the comparative Example 6 above).

### Example 9: New Process for the Preparation of Compound 45

As summarised in Table 9.1 below, the applicant has developed a new set of conditions for certain of the process steps used for synthesising compound 45, which compound can be used for the synthesis of the characteristic thiophene-based key amino intermediate 46. Applied together with the new reaction conditions for the preparation of compound 46 from compound 45 described in Example 8, the process to synthesise compound 45 represented by these new step-conditions shows advantages over the prior process for synthesising compound 46 as is described in PCT/EP2021/060338 (and as summarised below and described in the comparative Example 6 above), for example in terms of the overall scale of synthesis, yield and purity of the synthesis of compound 46, a key intermediate in the preparation of the SIK3 inhibitor compound E9.

TABLE 9.1
New conditions for certain steps of the process for synthesising
compound 45 compared to those described in PCT/EP2021/060338.

| | | Conditions described in |
|---|---|---|
| Step | New reaction conditions | PCT/EP2021/060338 |
| 33 & 34 to 35 | Ethyl-esters as educts; | Ethyl-esters as educts; |
| | Et₃N/EtOH | piperidine |
| 35 to 36 | Ethyl-esters as educts; | Ethyl-esters as educts; |
| | EtONa | NaH/THF |
| 36 to 37 | NH₂OH*HCl, MeCN | NH₂OH*HCl, MeCN |
| 37 to 38 | (Boc)₂O, dioxane, Na₂CO₃ | (Boc)₂O, dioxane, Na₂CO₃ |
| 38 to 39 | NCS, AcOH, THF | NCS, AcOH (no solvent) |
| 39 to 40 | LiAlH₄/THF | DIBAL/DCM |
| 40 to 45 | DAST/DCM | DAST/DCM |

1. A method of preparing the compound E9, especially a method of preparing greater than about 125 g of the compound E9 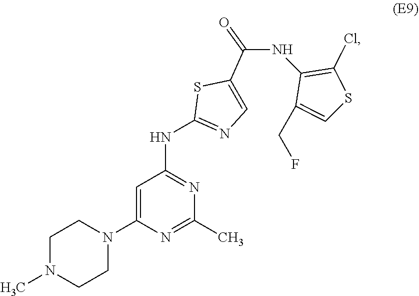 [SMILES: Cc1nc(Nc2ncc(C(=O)Nc3c(CF)csc3Cl)s2)cc(N2CCN(C)CC2)n1]
wherein the method comprises a step of reacting under coupling conditions a carboxylic acid intermediate having the formula I: 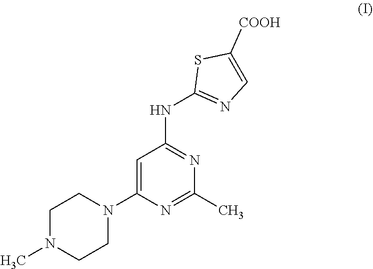 [SMILES: Cc1nc(Nc2ncc(C(=O)O)s2)cc(N2CCN(C)CC2)n1]
with an amino intermediate having the formula II: 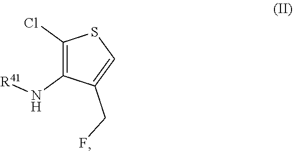
wherein R⁴¹ is selected from the group consisting of H or an amino protecting group.

2. The method of claim 1, wherein R⁴¹ is H, especially wherein the amino intermediate having formula II is compound 46.

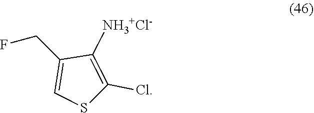

3. The method of claim 1, wherein the reaction of the amino intermediate II with carboxylic acid intermediate I is conducted in the presence of a coupling agent and/or a base.

4. The method of claim 2, wherein the reaction comprises:
reacting about 1 Equiv. of the carboxylic acid intermediate having formula I: 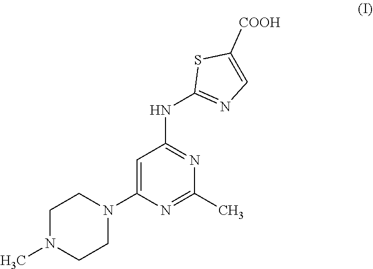 [SMILES: Cc1nc(Nc2ncc(C(=O)O)s2)cc(N2CCN(C)CC2)n1]
with between 0.5 and 5 Equiv. of the amino intermediate 46 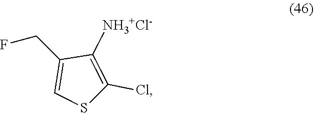
in the presence of between 0.8 and 7 Equiv. of tetramethylchloroformamidinium hexafluorophosphate (TCFH) and between 0.2 and 20 Equiv. of N,N-diisopropylethylamine (DiPEA).

5. The method of claim 4, wherein the carboxylic acid intermediate having formula I, the amino intermediate 46 and the tetramethylchloroformamidinium hexafluorophosphate (TCFH) are mixed in a polar aprotic solvent to form a mixture and the N,N-diisopropylethylamine (DiPEA) is added subsequently to the mixture.

6. The method of claim 5, wherein the N,N-diisopropylethylamine (DiPEA) is added to the mixture over a period of time of 2 min to 12 h.

7. The method of claim 1, further comprising a step of preparing the carboxylic acid intermediate having formula I: 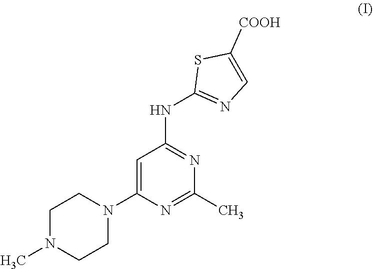 [SMILES: Cc1nc(Nc2ncc(C(=O)O)s2)cc(N2CCN(C)CC2)n1]
by hydrolysis of an ester intermediate having the formula Ta:

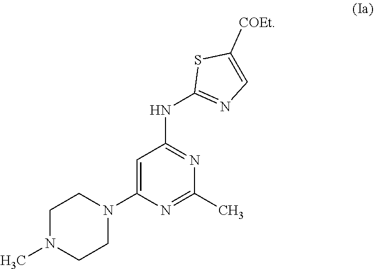

8. The method of claim 7, further comprising a step of preparing the ester intermediate having formula Ia by reacting under coupling conditions compound 3 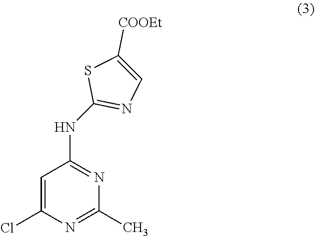 [SMILES: CCOC(=O)c1cnc(Nc2cc(Cl)nc(C)n2)s1]
with methylpiperazine.

9. The method of claim 1, wherein the amino intermediate is compound 46, and the method further comprises a step of preparing compound 46 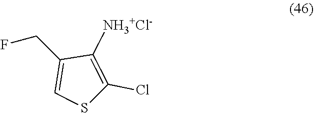
by reacting compound 45 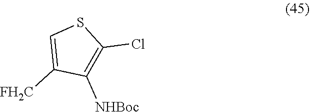
with HCl and ethyl acetate (EA).

10. A method of preparing an amino intermediate useful for preparing the compound E9 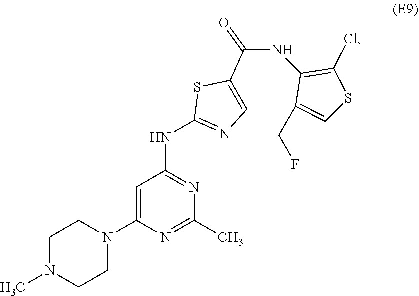 [SMILES: Cc1nc(Nc2ncc(C(=O)Nc3c(CF)csc3Cl)s2)cc(N2CCN(C)CC2)n1]
wherein the amino intermediate is 46 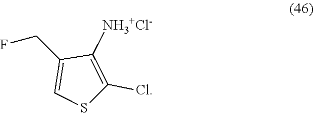
especially a method of preparing greater than about 2.5 g of the amino intermediate 46, wherein the method comprises a step of reacting compound 45 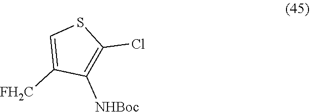
with HCl and ethyl acetate (EA).

11. The method of claim 10, wherein the reaction is conducted at between 5 and 40° C. for between 1 and 18 h.

12. The method of claim 10, wherein each about 1 Equiv. of compound 45 is reacted with about 4M HCl and about 10 Equiv. of ethyl acetate and the reaction is conducted at between 2° and 30° C. for about 18 h, or by reacting each about 1 Equiv. of compound 45 with about 4M HCl and about 4 Equiv. of ethyl acetate and the reaction is conducted at between 2° and 30° C. for about 4 h.

13. An intermediate useful for preparing the compound E9 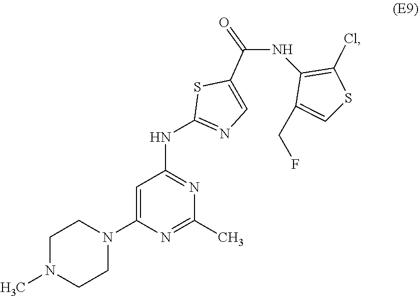 [SMILES: Cc1nc(Nc2ncc(C(=O)Nc3c(CF)csc3Cl)s2)cc(N2CCN(C)CC2)n1]
wherein the intermediate is a carboxylic acid intermediate having the formula I:

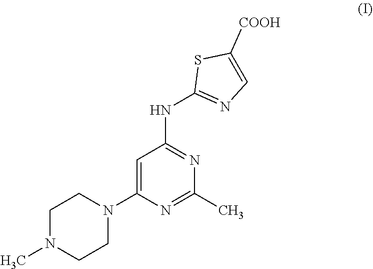 [SMILES: Cc1nc(Nc2ncc(C(=O)O)s2)cc(N2CCN(C)CC2)n1]

14. An intermediate useful for preparing the compound E9 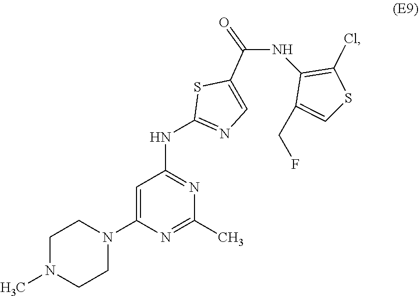 [SMILES: Cc1nc(Nc2ncc(C(=O)Nc3c(CF)csc3Cl)s2)cc(N2CCN(C)CC2)n1]
wherein the intermediate is an ester intermediate having the formula Ia:

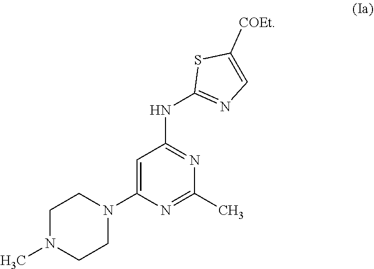

15. A bulk amount of a compound, wherein the compound is one and, in the respective amount, selected from the group consisting of:
(A) at least about 125 g of compound E9 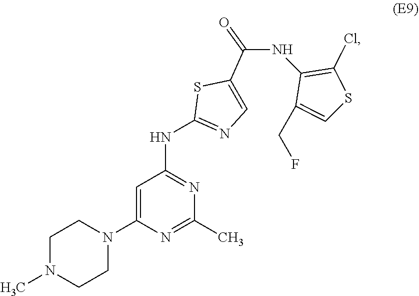 [SMILES: Cc1nc(Nc2ncc(C(=O)Nc3c(CF)csc3Cl)s2)cc(N2CCN(C)CC2)n1]
(B) at least about 125 g of the carboxylic acid intermediate having the formula I: 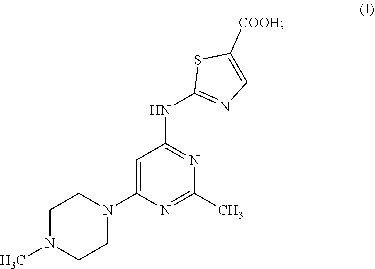 [SMILES: Cc1nc(Nc2ncc(C(=O)O)s2)cc(N2CCN(C)CC2)n1]
(C) at least about 125 g of the ester intermediate having the formula Ia: 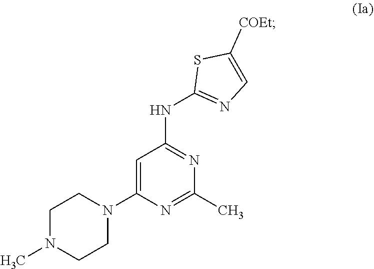
(D) at least about 2.5 g of the amino intermediate 46 [chemical structure]
